SEQUENCE LISTING (13 sequences)

SEQ ID NO: 1 - DNA, 23 bp, Artificial Sequence
  source 1..23: /mol_type=“unassigned DNA” /note=“16S forward primer” /organism=“Artificial Sequence”
atgatctagcttgctagattgat

SEQ ID NO: 2 - DNA, 19 bp, Artificial Sequence
  source 1..19: /mol_type=“unassigned DNA” /note=“16S reverse primer” /organism=“Artificial Sequence”
actgatcgtcgccttggtg

SEQ ID NO: 3 - DNA, 17 bp, Artificial Sequence
  source 1..17: /mol_type=“unassigned DNA” /note=“universal primer” /organism=“Artificial Sequence”
gtttgatcctggctcag

SEQ ID NO: 4 - DNA, 20 bp, Artificial Sequence
  source 1..20: /mol_type=“unassigned DNA” /note=“universal primer” /organism=“Artificial Sequence”
gcccgggaacgtattcaccg

SEQ ID NO: 5 - DNA, 30 bp, Artificial Sequence
  source 1..30: /mol_type=“unassigned DNA” /note=“forward nested primer” /organism=“Artificial Sequence”
cgcccggggcgcgccccgggcggggcgggg

SEQ ID NO: 6 - DNA, 27 bp, Artificial Sequence
  source 1..27: /mol_type=“unassigned DNA” /note=“reverse nested primer” /organism=“Artificial Sequence”
gcacggggggaacgcgaagaaccttac

SEQ ID NO: 7 - DNA, 1639 bp, Artificial Sequence
  source 1..1639: /mol_type=“unassigned DNA” /note=“N234_18635 16S ribosomal RNA” /organism=“Artificial Sequence”
ccgataagtgtgaacgctaggcttgagacacaagcctctggaaaggaggtgatccagccgcaccttccgatacggctaccttgttacgacttcaccccagtcatgaaccctgccgtggtaatcgccctccttgcggttaggctaactacttctggcaaaacccactcccatggtgtgacgggcggtgtgtacaagacccgggaacgtattcaccgcggcatgctgatccgcgattactagcgattccagcttcacgtagtcgagttgcagactacgatccggactacgatgcgttttctgggattagctccccctcgcgggttggcaaccctctgtacgcaccattgtatgacgtgtgaagccctacccataagggccatgaggacttgacgtcatccccaccttcctccggtttgtcaccggcagtctctctagagtgctcttgcgtagcaactaaagacaagggttgcgctcgttgcgggacttaacccaacatctcacgacacgagctgacgacagccatgcagcacctgtgtccactttccctttcgggcacctaatgcatctctgcttcgttagtggcatgtcaagggtaggtaaggtttttcgcgttgcatcgaattaatccacatcatccaccgcttgtgcgggtccccgtcaattcctttgagttttaatcttgcgaccgtactccccaggcggtcaacttcacgcgttagctacgttactgaagaaatgaatccccaacaactagttgacatcgtttagggcgtggactaccagggtatctaatcctgtttgctccccacgctttcgtgcatgagcgtcagtgacgtcccagggggctgccttcgccatcggtattcctccacatctctacgcatttcactgctacacgtggaattctacccccctctgacatactctagccttgcagtcacaagcgccattcccaagttaagctcggggatttcacgcctgtcttacaaaaccgcctgcgcacgctttacgcccagtaattccgattaacgctcgcaccctacgtattaccgcggctgctggcacgtagttagccggtgcttattcttccggtaccgtcatcgacccggggtattatcccaggccatttctttccggacaaaagtgctttacaacccgaaggccttcttcacacacgcggcattgctggatcagggttgcccccattgtccaaaattccccactgctgcctcccgtaggagtctgggccgtgtctcagtcccagtgtggctgatcgtcctctcagaccagctactgatcgtcgccttggtaggctcttaccccaccaactagctaatcagacatcggccgctcctgtagcgcgaggccttgcggtcccccgctttcaccctcaggtcgtatgcggtattagctaatctttcgactagttatcccccactacagggcacgttccgatgtattactcacccgttcgccactcgccaccaggccgaagcccgtgctgccgttcgacttgcatgtgtaaggcatgccgccagcgttcaatctgagccaggatcaaactcttcagttcaatctctgtgtggaccctcgcgggtcctcgctctttcgagcggtcgctcactctcagaaaactgac

SEQ ID NO: 8 - DNA, 21 bp, Artificial Sequence
  source 1..21: /mol_type=“unassigned DNA” /note=“forward primer Ralstonia mannolitytica” /organism=“Artificial Sequence”
gggaaagcttgctttcctgcc

SEQ ID NO: 9 - DNA, 22 bp, Artificial Sequence
  source 1..22: /mol_type=“unassigned DNA” /note=“reverse primer Ralstonia mannolitytica” /organism=“Artificial Sequence”
tccgggtattaaccagagccat

SEQ ID NO: 10 - DNA, 23 bp, Artificial Sequence
  source 1..23: /mol_type=“unassigned DNA” /note=“Forward primer Ralstonia insidiosa” /organism=“Artificial Sequence”
atgatctagcttgctagattgat

SEQ ID NO: 11 - DNA, 23 bp, Artificial Sequence
  source 1..23: /mol_type=“unassigned DNA” /note=“reverse primer Ralstonia insidiosa” /organism=“Artificial Sequence”
cacacctaatattagtaagtgcg

SEQ ID NO: 12 - DNA, 21 bp, Artificial Sequence
  source 1..21: /mol_type=“unassigned DNA” /note=“Forward primer Ralstonia eutropha” /organism=“Artificial Sequence”
accccggggtcgatgacggta

SEQ ID NO: 13 - DNA, 20 bp, Artificial Sequence
  source 1..20: /mol_type=“unassigned DNA” /note=“reverse primer Ralstonia eutropha” /organism=“Artificial Sequence”
gccttgcagtcacaagcgcc

(19) United States
(12) Patent Application Publication
(10) Pub. No.: US 2017/0252421 A1
(43) Pub. Date: Sep. 7, 2017

(54) COMPOUNDS AGAINST RALSTONIA PICKETTII FOR USE IN THE TREATMENT OF INSULIN RESISTANCE, AND METHOD OF DIAGNOSIS OF INSULIN RESISTANCE

(71) Applicants: ACADEMISCH MEDISCH CENTRUM, Amsterdam (NL); WAGENINGEN UNIVERSITEIT, Wageningen (NL)

(72) Inventors: Max NIEUWDORP, Amsterdam (NL); Willem Meindert DE VOS, Wageningen (NL)

(73) Assignees: ACADEMISCH MEDISCH CENTRUM, Amsterdam (NL); WAGENINGEN UNIVERSITEIT, Wageningen (NL)

(21) Appl. No.: 15/506,948

(22) PCT Filed: Aug. 28, 2015
(86) PCT No.: PCT/EP2015/069733

§ 371 Date: Feb. 27, 2017

(30) Foreign Application Priority Data
Aug. 28, 2014 (EP) 14182707.1

(51) Int. Cl. A61K 39/02; C07K 16/12

(52) U.S. Cl. CPC A61K 39/0208; C07K 16/1203; C07K 2317/76

(57) ABSTRACT

Compounds disclosed herein are effective against Ralstonia pickettii for treating or preventing insulin resistance, obesity or type II diabetes of a subject, and include an antibiotic effective against Ralstonia pickettii, an immunogenic compound producing a protective immune response, or an antibody which specifically binds to Ralstonia pickettii or a fragment thereof. In vitro methods for diagnosis or prediction of insulin resistance, obesity or type II diabetes include determining the presence of Ralstonia pickettii or of an antibody which specifically binds to Ralstonia pickettii in a test sample. An antibody binding specifically to an antigen of Ralstonia pickettii, a Ralstonia pickettii cell, or a nucleic acid hybridizing under stringent conditions to a nucleic acid from Ralstonia pickettii is disclosed. A kit including said antibody, the nucleic acid, and optionally a Ralstonia pickettii bacteria or a nucleic acid or protein thereof, a further reagent or a conventional kit component, is also disclosed.

### TECHNICAL FIELD

The invention relates to compounds effective against Ralstonia pickettii, such as immunogenic compounds, antibodies and antibiotics for use in the treatment or prevention of insulin resistance, obesity, type II diabetes, metabolic syndrome, non-alcoholic fatty liver disease (NAFLD) or non-alcoholic steatosis Hepatitis (NASH) of a subject. The invention further relates to pharmaceutical compositions for use in the treatment of insulin resistance, obesity, type II diabetes, and/or their complications.

The invention further relates to a method of diagnosis or prediction of insulin resistance, obesity or type II diabetes in a subject, comprising determining the presence of Ralstonia pickettii or the presence of an antibody which specifically binds to Ralstonia pickettii in a test sample. More in particular, the invention relates to subjects who suffer from visceral fat inflammation.

The invention also relates to compounds and reagents and kits of parts for use in the diagnostic method.

### BACKGROUND

Obesity is a condition characterized by an excess of body fat. The prevalence of overweight and obesity is considered an important public health issue in the world. Roughly two thirds of US adults meet the criteria for overweight or obesity. Actually, obesity is an important risk factor for cardiovascular disease (CVD), ventricular dysfunction, congestive heart failure, stroke, and cardiac arrhythmias. Furthermore obesity is closely associated with type 2 diabetes, metabolic syndrome, non-alcoholic fatty liver disease (NAFLD) and Non-Alcoholic Steatosis Hepatitis (NASH).

Type 2 diabetes mellitus (DM2), or non insulin-dependent diabetes mellitus (NIDDM), is characterized by the fact that patient produce insulin and even exhibit hyperinsulinemia (plasma insulin levels that are the same or even elevated in comparison with non-diabetic subjects), while at the same time demonstrating hyperglycemia. Subjects with DM2 often develop “insulin resistance”, such that the effect of insulin in stimulating glucose and lipid metabolism in the main insulin-sensitive tissues, namely, muscle, liver and adipose tissues, is diminished and those patients are thus at increased risk of cardiovascular complications, e.g. atherosclerosis, coronary heart disease, stroke, peripheral vascular disease, hypertension, nephropathy, neuropathy and retinopathy.

About 33% of all obese patients develop insulin resistance, but have not yet developed type 2 diabetes (malign obesity), and these subjects are also at a risk of developing metabolic syndrome. Metabolic syndrome represents a pre-stage of type 2 diabetes and often precedes cardiovascular diseases (CVD). It is characterized by insulin resistance, along with abdominal obesity, hyperinsulinemia, high blood pressure and low High Density Lipoprotein (HDL) and high Very Low Density Lipoprotein (VLDL)/triglyceride plasma cholesterol levels. In the medical field there appears to be consensus that the term metabolic syndrome is acceptable for the condition of the presence of multiple metabolic risk factors for CVD and type 2 diabetes. Patients with the metabolic syndrome have a doubled risk of developing CVD over the next 5 to 10 years as compared to obese individuals without the syndrome. Also, the metabolic syndrome confers a 5-fold increase in risk for type 2 diabetes. Metabolic syndrome can be clinically diagnosed by the presence of any 3 of the following 5 risk factors: elevated waist circumference, elevated triglycerides, reduced HDL-C, elevated blood pressure, and elevated fasting glucose (Alberti et al. 2009. Circulation 120:1640-1645).

With more obesity in the developed world, the prevalence of hepatic steatosis, or fatty liver, has increased. An estimated ˜1/3 of the population in the United States is obese (BMI>30) and ˜40% have type 2 diabetes or prediabetes (http://www.cdc.gov/diabetes/pubs/pdf/ndfs_2011.pdf). In response to overtaxed adipose tissues, human livers attempt to compensate as a storage depot for triglycerides (TGs). This has resulted in an epidemic of nonalcoholic fatty liver disease (NAFLD), with a prevalence of up to 30% in the United States. Excessive fat in the liver is associated with organ pathology, including nonalcoholic steatohepatitis (NASH) and cirrhosis, which can lead to liver failure.

Nonalcoholic hepatic steatosis is usually found in obese subjects with metabolic syndrome. Recent studies have documented a strong relationship between hepatic steatosis and insulin resistance. The prevalence of other metabolic abnormalities associated with insulin resistance such as abnormal glucose tolerance, hypertriglyceridemia, and low levels of high-density lipoprotein cholesterol is also high in subjects with NAFLD. Metformin is considered a first-line treatment in the management of insulin resistance and type 2 diabetes mellitus. Metformin is often used to decrease fasting and postprandial hyperglycemia in combination with modifications to the diet and lifestyle changes. Metformin can also decrease triglycerides levels and has also been associated with a modest weight loss.

Adverse reactions are frequent in patients taking metformin. The most notable are the gastrointestinal side effect such as anorexia, nausea, vomiting, abdominal discomfort and diarrhoea. Up to 20% of patients taking oral metformin will experience one of these side effects. The effects are dose related however, up to 5% will discontinue the therapy due to the side effects. 77% of patients taking metformin will also develop a vitamin B12 deficiency. Vitamin B12 deficiency has been associated with peripheral neuropathy, a common finding among type 2 diabetics. At present, treating insulin resistance in these subjects with metformin reduces morbidity and mortality rates by about 30%.

Vrieze et al. (2013. The role of gut microbiota in human metabolism) investigated whether patients suffering from obesity but otherwise healthy with chronic visceral adipose tissue inflammation were characterized by increased endotoxemia and altered intestinal microbiota composition. From a set of 47 patients having a BMI between 18-45 kg/m2, 12 patients with the highest lipopolysaccharide binding protein (LBP) plasma levels and 12 patients with the lowest LBP plasma levels were selected, and several parameters of these groups of patients were studied (Table 1). It was found that patients in the high LBP group had a higher BMI, higher leucocyte count, and a higher CRP compared to the low LBP group. No significant difference between the composition of the faecal communities of these two groups could be detected. Additionally, the authors checked bacterial DNA in visceral adipose tissue. Bacteria related to the gram-negative Ralstonia species were identified to be the ubiquitous bacterial species in visceral adipose tissue. The amount of Ralstonia DNA was significantly increased in subjects with low LBP levels compared to the high LBP group. Thus, patients with high BMI had lower amount of Ralstonia DNA.

WO2012/131099 is concerned with the use of bacterial flora for vaccine development, identification of therapeutic targets and prediction and/or diagnosis of metabolic diseases such as diabetes, overweight and obesity, and their CVD complications. 5212 adults aged 30-65 years at baseline were recruited, which were clinically and biologically evaluated for development of diabetes at inclusion and at 3-, 6-, and 9-year follow-up visits. 16S rDNA was sequenced in the pooled DNA sample from cases of incident diabetes and from controls without diabetes over the entire follow-up period. It was found that the level of many intestinal bacterial phyla, families and genera was different in healthy subjects as compared to overweight, obese, or morbidly obese subjects. It was suggested that any one of these intestinal bacterial phyla, families and genera could be used as an immunogenic or vaccinal composition, particularly for preventing onset of metabolic diseases such as diabetes, overweight and obesity, and their CVD complications. However, it is unlikely that immunogenic or vaccinal composition based on any phyla, families and genera mentioned is useful for preventing onset of metabolic diseases. In actual fact, the only vaccine composition tested was a vaccine consisting of ultrasonically inactivated bacteria (ileum mix) originating from mice rendered diabetic by a high fat diet, without any identification of the phyla, families and genera present in such inactivated mix.

There is a need for alternatives for treating and/or preventing insulin resistance, obesity or obesity-related disorders such as DM2, metabolic syndrome and non-alcoholic fatty liver diseases (NAFLD/NASH).

### SUMMARY

The invention is based on the surprising finding that cells of Ralstonia pickettii, a Gram-negative bacterium, were identified in test samples of patients suffering from obesity (both in faeces and mesenteric visceral adipose tissue). In addition, the inventors have shown that the concentrations of Ralstonia pickettii were correlated with both clinical and epidemiological features of insulin resistance. Moreover, 4 weeks daily gavage with Ralstonia pickettii bacteria induced insulin resistance in diet induced obesity (DIO) mice. The inventors therefore believe that the species Ralstonia pickettii might thus be the potential initiator of chronic inflammation linking insulin resistance to malign obesity via intestinal microbiota composition. The inventors have also shown that treatment against Ralstonia pickettii is effective in reversing the clinical effects of insulin resistance. Targeted pre-treatment of DIO mice using either exogenous polyclonal antibodies or vaccination against Ralstonia pickettii could reverse insulin resistance. Immunoboosting against intestinal Ralstonia pickettii significantly affected level of insulin resistance and subsequent visceral-mesenteric adipose tissue inflammation in DIO mice.

In a first aspect, the invention teaches a compound effective against Ralstonia pickettii for use as a medicament. Preferably, said compound is selected from the group consisting of an antibiotic effective against Ralstonia pickettii, an immunogenic compound derivable from Ralstonia pickettii, preferably an antigen derived from R. pickettii, which is capable of inducing an immune response to a Ralstonia pickettii infection in a subject, an Immunoglobulin (Ig)-like molecule, preferably an antibody, which is capable of neutralizing Ralstonia pickettii, or a binding fragment thereof, and a bacteriophage specific for Ralstonia pickettii.

In a further aspect, the invention provides the compound as described above for use in the treatment or prevention of insulin resistance, obesity, type II diabetes, metabolic syndrome, non-alcoholic fatty liver disease (NAFLD) or Non-alcoholic Steatosis Hepatitis (NASH) of a subject.

The invention further provides a composition comprising a compound as defined above and a pharmaceutically-acceptable excipient, carrier, or diluent for use in the treatment or prevention of insulin resistance, obesity, type II diabetes, metabolic syndrome, non-alcoholic fatty liver disease (NAFLD) or Non-Alcoholic Steatosis Hepatitis (NASH) of a subject.

The invention further provides a vaccine composition comprising Ralstonia pickettii bacterium which is live, inactivated, or attenuated, or a fragment thereof capable of producing a protective immune response in an animal. Preferably, said vaccine composition is a conventional vaccine.

Preferably, said vaccine composition is for use in the treatment or prevention of insulin resistance, obesity, type II diabetes, metabolic syndrome, non-alcoholic fatty liver disease (NAFLD) or Non-Alcoholic Steatosis Hepatitis (NASH) of a subject.

In an embodiment, the compound taught herein is an antibiotic selected from the group consisting of ceftriaxone, imipenem, cilastatin, piperacillin, tazobactam, amikacin, gentamicin, cefoperazone-sulbactam, ciprofloxacin, colistine, and cefotaxime. Colistine may be administered orally. Cefotaxime and imipenem may be administered intravenously. In an embodiment, said composition comprises nanoparticles.

In a further aspect, the invention provides an in vitro method of diagnosis or prediction of a disease selected from insulin resistance, obesity or type II diabetes, metabolic syndrome, non-alcoholic fatty liver disease (NAFLD) or Non-alcoholic Steatosis Hepatitis (NASH), in a subject, comprising steps of: determining the level of Ralstonia pickettii or the level of an antibody capable of neutralizing Ralstonia pickettii in a test sample derived from said subject, comparing said presence with a reference value or the level of Ralstonia pickettii or the level of an antibody capable of neutralizing Ralstonia pickettii in a control sample, and diagnosing or predicting said disease based on said comparison.

Preferably, said subject is a patient suffering from visceral fat inflammation, preferably mesenteric visceral fat inflammation. In a preferred embodiment, said test sample is selected from the group consisting of a faeces sample or a sample comprising adipose cells, preferably from visceral adipose fat, more preferably mesenteric visceral adipose fat as presence of the bacterium in these samples is highly indicative of the disease. Preferably, said method comprises determining the presence of bacterial nucleic acid of Ralstonia pickettii in a test sample of said subject, preferably 16S rRNA.

In another aspect, the invention provides the use of an antibody capable of neutralizing Ralstonia pickettii, a Ralstonia pickettii cell, and/or a nucleic acid hybridizing under stringent conditions to a nucleic acid from Ralstonia pickettii in a method of diagnosis or prediction as taught herein.

In a further aspect, the invention provides the use of a kit comprising said antibody, a nucleic acid as defined above, or a Ralstonia pickettii bacterium or a nucleic acid or protein thereof, and optionally further comprising a further reagent or a conventional kit component in a method of diagnosis or prediction as taught herein.

### BRIEF DESCRIPTION OF THE DRAWINGS

FIG. 1 shows the presence of bacterial DNA in mesenteric-visceral adipose tissue from otherwise healthy obese subjects that underwent laparoscopic surgery. (A) Pyrosequencing revealed the presence of different species (percentage of total bacterial DNA) in human mesenteric visceral adipose tissue specimen (n=12 subjects) with Ralstonia spp. being the most abundant Gram-negative bacteria. (B) A correlation between mesenteric visceral Ralstonia spp. DNA content and HOMA-IR, (C) plasma adiponectin and (D) fecal R. pickettii was found. Also, (E) fecal R. pickettii was correlated with HOMA-IR (Pearsons correlation coefficient).

FIG. 2 shows (A) Predictive value of fecal 16S rRNA Ralstonia Pickettii levels in the development of type 2 diabetes (T2D) (n=47), impaired glucose tolerance (IGT) (n=45) and normal glucose tolerance (NGT) (n=42) in obese postmenopausal women, and (B) relation between plasma adiponectin and fecal Ralstonia pickettii levels.

FIG. 3 shows the effects of R. pickettii treatment and prevaccination treatment in male DIO C57B16 mice (n=9-15 mice per treatment group), on (A) fecal R. pickettii concentrations, (B) mesenteric adipose tissue R. pickettii concentrations, (C) weight, (D) food intake, (E) oral glucose tolerance (OGTT), and (F) iAUC of OGTT.

FIG. 4 shows in vitro dose-dependent effect of IgG polyclonal antibodies (generated in rabbits) on binding efficacy of increasing dosages of Ralstonia pickettii.

FIG. 5 shows the effects of R. pickettii treatment in male DIO C57B16 mice (n=9-15 mice per treatment group), on (a) FASN/fatty acid synthase, (b) ACC/Acetyl CoA, and (c) MRP2/ATP Binding Cassette Protein C2 hepatic gene expression in 4 treatment groups using glycerol, active R. pickettii, heat inactivated R. pickettii and prevaccination with R. pickettii extract. Variance was similar between the groups that were statistically compared.

### DETAILED DESCRIPTION

### Definitions

As used herein, the term “Ralstonia pickettii” refers to a Gram-negative, rod-shaped bacterium belonging to the beta-Proteobacteria found in moist environments such as soils, river and lakes. A taxonomic overview is provided in IJSEM July 2003 vol. 53 no. 4 1075-1080. Ralstonia pickettii include the species RefSeq (Assembly:GCF_000023425) Ralstonia pickettii 12D and RefSeq (Assembly:GCF 000020205) Ralstonia pickettii 12J. Samples of Ralstonia pickettii are deposited or available in DSM No.: 6297, ATCC 27511, CIP 73.23, JCM 5969, NCTC 11149. The draft genome of Ralstonia pickettii AU12-08 has been deposited in NCBI GenBank under the accession no. ASZV00000000.

As used herein, the term “insulin resistance” refers to the inability of a patient's cells to respond to insulin appropriately or efficiently. The pancreas responds to this problem at the cellular level by producing more insulin. Eventually, the pancreas cannot keep up with the body's need for insulin and excess glucose builds up in the bloodstream. Patients with insulin resistance often have high levels of blood glucose and high levels of insulin circulating in their blood at the same time.

As used herein the term “obesity” has its commonly understood meaning such as “excessively fat” and includes the clinical designation of being obese as defined in and by the medical literature and brochures of support or public health organizations. For example, Dorland's Illustrated Medical Dictionary (29th edition, W.B. Saunders Company, Philadelphia USA.) defines obesity as an increase in bodyweight beyond the limitation of skeletal and physical requirements, as the result of an excessive accumulation of fat in the body.” Because the decision of suitability for treatment of a patient with compound(s) of the present invention to a patient is to be made by a qualified physician or care giver, the patient is inherently deemed suitable or obese by the administering caregiver.

The term “type II diabetes” or “type 2 diabetes” as used herein refers to non-insulin-dependent diabetes. Type II diabetes refers to an insulin-related disorder in which there is a relative disparity between endogenous insulin production and insulin requirements, leading to elevated hepatic glucose production, elevated blood glucose levels, inappropriate insulin secretion, and peripheral insulin resistance. Type II diabetes has been regarded as a relatively distinct disease entity, but type II diabetes is often a manifestation of a much broader underlying disorder (Zimmet et al (2001) Nature 414: 782-787), which may include metabolic syndrome (syndrome X), diabetes (e.g., type I diabetes, type II diabetes, gestational diabetes, autoimmune diabetes), hyperinsulinemia, hyperglycemia, impaired glucose tolerance (IGT), hypoglycemia, B-cell failure, insulin resistance, dyslipidemias, atheroma, insulinoma, hypertension, hypercoagulability, microalbuminuria, and obesity and obesity-related disorders such as visceral obesity, central fat, obesity-related type II diabetes, obesity-related atherosclerosis, heart disease, obesity-related insulin resistance, obesity-related hypertension, microangiopathic lesions resulting from obesity-related type II diabetes, ocular lesions caused by microangiopathy in obese individuals with obesity-related type II diabetes, and renal lesions caused by microangiopathy in obese individuals with obesity-related type II diabetes.

Some of the more common adult onset diabetes symptoms include fatigue, excessive thirst, frequent urination, blurred vision, a high rate of infections, wounds that heal slowly, mood changes and sexual problems. Despite these known symptoms, the onset of type II diabetes is often not discovered by health care professionals until the disease is well developed. Once identified, type II diabetes can be recognized in a patient by measuring fasting plasma glucose levels and/or casual plasma glucose levels, measuring fasting plasma insulin levels and/or casual plasma insulin levels, or administering oral glucose tolerance tests or hyperinsulinemic-euglycemic clamp tests.

As used herein the term “insulin resistance” refers to a state in which cells of a subject fail to respond to normal levels of circulating insulin. At the molecular level insulin resistance relates to abnormally reduced signalling and metabolic outcome (e.g., glucose uptake, metabolism, or storage) through the insulin receptor pathway. Insulin resistance is associated with numerous medical conditions (i.e., causes or is a consequence of) including, but not limited to, diabetes (e.g., Type 2 diabetes), obesity, hyperglycemia, hyperlipidemia, acute trauma, chronic infection and cardiovascular diseases.

The term “antibiotic” as used herein, in accordance with its accepted meaning, refers to a substance produced during the growth of a microorganism and which in high dilution is antagonistic to one or more other microorganisms when added to media in which the latter normally grow.

As used herein, the term “immunogenic” refers to the ability of a compound to elicit an immune response.

The phrases “specifically binds” when referring to a protein, when referring to an antibody, refers to a binding reaction which is determinative of the presence of the protein in the presence of a heterogeneous population of proteins and other biological molecules. Thus, under designated conditions, a specified ligand binds preferentially to a particular protein and does not bind in a significant amount to other proteins present in the sample. A molecule or ligand (e.g., an antibody) that specifically binds to a protein has an association constant of at least 10³ M⁻¹ or 10⁴ M⁻¹ sometimes 10⁵ M⁻¹ or 10⁶ M⁻¹, in other instances 10⁶ M⁻¹ or 10⁷ M⁻¹ preferably 10⁸ M⁻¹ to 10⁹ M⁻¹, and more preferably, about 10¹⁰ M′ to 10¹¹ M⁻¹ or higher.

The term “pharmaceutically-acceptable excipient, carrier, or diluent” as used herein means a pharmaceutically-acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the composition and not injurious to the patient.

The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like.

The term “pharmaceutically acceptable” is employed herein to refer to those compounds, materials, compositions, and/or dosage forms which are, within the scope of sound medical judgment, suitable for use in contact with the tissues of human beings, animals and plants without excessive toxicity, irritation, allergic response, or other problem or complication, commensurate with a reasonable benefit/risk ratio.

The term “inactivated bacteria” as used herein, refers to Ralstonia pickettii bacteria that are incapable of infection and/or colonization and encompasses attenuated as well as killed bacteria. “Attenuated bacteria” may replicate but cannot cause infection or disease. Inactivation of bacteria may be accomplished by any methods known by those skilled in the art. For example, the bacteria may be chemically inactivated, such as by formalin fixation, or physically inactivated such as by heat, sonication or irradiation, so that they are rendered incapable of replication and/or infection and/or causing disease.

It is preferred that an effective amount of the active vaccine is administered, in which “effective amount” is defined as an amount of Ralstonia pickettii bacteria or an immunogenic fragment or derivative thereof that is capable of producing an immune response in a subject. The amount needed will vary depending upon the antigenicity of the antigen, such as bacteria, fragment, or derivative, used, and the species and weight of the subject to be vaccinated, and may be ascertained using standard techniques. In preferred, non-limiting embodiments of the invention, an effective amount of vaccine produces an elevation of antibacterial antibody titre to at least two times the antibody titre prior to vaccination. In a preferred, specific, non-limiting embodiment of the invention, approximately 10⁷ to 10¹¹ bacteria and preferably 10⁸ to 10¹⁰ bacteria are administered to a host.

The term “conventional vaccine”, as used herein, is meant to define well-established vaccines, whether they are based on (1) live, attenuated vaccine components (not adjuvanted), or (2) killed inactivated vaccine components, killed whole cells, purified subunit or peptide components (which are adjuvanted), using e.g. mineral adjuvants.

The term “effective amount” as applied to passive vaccines is an amount of antibody that is capable of preventing or attenuating a bacterial disease or infection. The amount needed will vary depending upon the type of antibody and the antibody titre, and the species and weight of the subject to be vaccinated, but may be ascertained using standard techniques.

The term “visceral fat” or “visceral adipose tissue” as used herein has its ordinary meaning as understood by those skilled in the art and includes the fat in the abdominal region which is inside the peritoneal cavity, and thus is distinct from “subcutaneous fat”. Visceral fat can be assessed, either qualitatively or quantitatively, by standard assays known to those of ordinary skill in the art, for example, by computer tomography (CT), magnetic resonance imaging (MRI), ultrasonography, and/or determinations of subject waist-to-hip measurement ratios.

As used herein, the terms “adipocyte” or “adipose cell” encompass both white adipose cells and brown adipose cells.

As used herein, “16S DNA” refers to the gene encoding the 16S ribosomal RNA (16S rRNA) constituted of about 1500 nucleotides, which is the main component of the small prokaryotic ribosomal subunit (30S). 16S DNA is highly conserved among bacteria. The reference Ralstonia pickettii 16S rRNA gene sequence corresponds to SEQ ID NO: 7. In the context of the invention, 16S DNA refers to any sequence corresponding to SEQ ID NO: 7 in other bacterial strains.

As used herein, a “diagnosing method” or “diagnostic method” or “diagnosis” refers to a method for determining whether an individual suffers from a disease.

As used herein, a “predicting method” refers to a method for determining whether an individual is likely to develop a disease.

In the context of the present invention, a “subject” denotes a human or non-human mammal, such as a rodent (rat, mouse, and rabbit), a primate (chimpanzee), a feline (cat), a canine (dog). Preferably, the subject is human.

As used herein, the term “test sample” or “biological sample” means a substance of biological origin. Examples of test samples include, but are not limited to, blood and components thereof such as serum, plasma, platelets, subpopulations of blood cells such as leucocytes; faeces, and tissues such as adipose tissues, hepatic tissues and the like.

In the context of the invention, the terms “hybridize” or “hybridization” as is known to those skilled in the art, refer to the binding of a nucleic acid molecule to a particular nucleotide sequence under suitable conditions, namely under stringent conditions.

The term “stringent conditions” or “high stringency conditions” as used herein corresponds to conditions that are suitable to produce binding pairs between nucleic acids having a determined level of complementarity, while being unsuitable to the formation of binding pairs between nucleic acids displaying a complementarity inferior to said determined level. Stringent conditions are the combination of both hybridization and wash conditions and are sequence dependent. These conditions may be modified according to methods known from those skilled in the art (Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, high stringency conditions are selected to be about 50 C lower than the thermal melting point (Tm), preferably at a temperature close to the Tm of perfectly base-paired duplexes (Andersen, Nucleic acid Hybridization, Springer, 1999, p. 54). Hybridization procedures are well known in the art and are described for example in Ausubel, F. M., Brent, R., Kingston, R. E., Moore, D. D., Seidman, J. G., Smith, J. A., Struhl, K. eds. (1998) Current protocols in molecular biology. V. B. Chanda, series ed. New York: John Wiley & Sons.

High stringency conditions typically involve hybridizing at about 50° C. to about 68° C. in 5×SSC/5×Denhardt's solution/1.0% SDS, and washing in 0.2×SSC/0.1% SDS at about 60° C. to about 68° C. As used herein, the expression “hybridizes specifically” indicates that the nucleic acid of a determined sequence displays a sufficient degree of complementarity with a target sequence to form a stable binding between the nucleic acid and the target sequence and to avoid non-specific binding with a non-target sequence, under high stringency conditions. The degree of complementarity is calculated by comparing the sequence of said nucleic acid optimally aligned with the complementary target sequence, determining the number of positions at which the nucleic acid bases are complementary to yield the number of complementary positions, dividing the number of complementary positions by the total number of positions of the target sequence, and multiplying the result by 100 to yield the degree of complementarity. The degree of complementarity may be determined routinely using BLAST programs (basic local alignment search tools) and PowerBLAST programs known in the art (Altschul et al., (1990) J. MoI. Biol. 215: 403-410; Zhang and Madden (1997) Genome Res. 7:649-656).

As used herein, a “probe” refers to an oligonucleotide capable of binding in a base-specific manner to a complementary strand of nucleic acid. Probes according to the invention may be purified or recombinant. They may be labelled with a detectable moiety, i.e. a moiety capable of generating a detectable signal, such as radioactive, calorimetric, fluorescent, chemiluminescent or electrochemiluminescent signal. Numerous such detectable moieties are known in the art. By way of example, the moiety may be a radioactive compound or a detectable enzyme {e.g. horseradish peroxidase (HRP)). Preferably, the probe according to the invention comprises or is constituted of from about 10 to about 1000 nucleotides.

As used herein, the term “primer” refers to an oligonucleotide which is capable of annealing to a target sequence and serving as a point of initiation of DNA synthesis under conditions suitable for amplification of the primer extension product which is complementary to said target sequence.

### Embodiments

### Compounds

In a first aspect, the invention provides a compound effective against Ralstonia pickettii for use in the treatment or prevention of insulin resistance, obesity, type II diabetes, metabolic syndrome, non-alcoholic fatty liver disease (NAFLD) or non-alcoholic steatosis Hepatitis (NASH) of a subject. Any compound which is effective against Ralstonia pickettii may be used. The term “compound” as used herein means any pharmaceutical compound, i.e. drugs, prodrugs, or compounds otherwise indicated for animal use, as well as pharmaceutically acceptable salts and enantiomers of the foregoing. The term “effective” as used in this context refers to the ability of said compound to have an antibacterial effect on Ralstonia pickettii bacteria cells, preferably by limiting its growth or by killing it. Preferably, effective refers to the ability to reduce levels of viable Ralstonia pickettii bacteria and/or reduce viability of Ralstonia pickettii bacteria and/or reduce reproducibility of Ralstonia pickettii bacteria. Methods to test the effectiveness of a compound, e.g., the bacteriostatic or bactericidal effects of a compound, on specific bacteria are known in the art. A skilled person can therefore readily and without undue experimentation test the effectiveness of a certain compound against Ralstonia pickettii. In a preferred embodiment, said compound inhibits intestinal translocation of Ralstonia pickettii. Bacterial translocation as used herein is defined as the passage of viable bacteria from the gastrointestinal (GI) tract through the mucosal epithelium to other sites, such as the mesenteric lymph nodes, spleen, liver and blood.

Preferably, the effective compound is a compound that can be used in a treatment in a safe and effective amount in a subject. The term “safe and effective amount”, as used herein, means an amount of a compound or composition high enough to significantly positively modify the infectious condition being treated, but low enough to avoid serious side effects (at a reasonable benefit/risk ratio), within the scope of sound medical judgment. Compounds effectively used in the treatment of Ralstonia pickettii known in the art include antibiotics. Preferred antibiotics which are effective in the treatment of Ralstonia pickettii are described in Mikrobiyol Bul. 2009 April; 43(2):331-4. Preferred antibiotics include ceftriaxone, imipenem, cilastatin, piperacillin, tazobactam, amikacin, gentamicin, cefoperazone-sulbactam and ciprofloxacin colistine, and cefotaxime. In preferred embodiments, a dose of colistine is administered orally or a dose of cefotaxime and imipenem is administered intravenously, preferably using nanoparticles.

The inventors have shown herein that also compounds eliciting active and passive immunity are effective in the treatment or prevention of diseases according to the invention. Therefore, the compound of the invention encompasses antigens derived from Ralstonia pickettii and antibodies which are capable of neutralizing Ralstonia pickettii.

In another preferred embodiment, said compound is an immunogenic compound capable of producing a protective immune response in a subject, also known as an antigen. The inventors have shown that fragments of Ralstonia pickettii cells are capable of producing a protective immune response. In a preferred embodiment, said antigen is derived from an inactivated Ralstonia pickettii cell or a fragment thereof. A skilled person is capable of producing antigens based on Ralstonia pickettii cells or immunogenic fragments thereof. Live, attenuated or inactivated Ralstonia pickettii cells may be used as a source for the production of said immunogenic compound. Alternatively, live, attenuated or inactivated Ralstonia pickettii cells may be used as an antigen.

In aspects of the invention, Ralstonia pickettii cells are inactivated using formalin or by inactivating the bacteria using a heat treatment. Preferably, said Ralstonia pickettii cells are inactivated using a sonication method. Sonicating bacteria for vaccination purposes is a well-established technique and described in US patents U.S. Pat. No. 3,862,313, U.S. Pat. No. 4,298,597, and U.S. Pat. No. 4,707,543. Preferably, the bacterial cells are formalin killed prior to sonication, as described in U.S. Pat. No. 3,862,313.

The bacterial outer membrane of Ralstonia pickettii is believed to provide an interface for host-pathogen interactions and harbours proteins and polysaccharides that have a variety of important roles including the adherence to and invasion of host cells, resistance to phagocytosis and the degradation of host cells. Surface associated proteins also play a role in maintaining structural integrity and in the adaptation of bacterial pathogens to differing environments within the host. As they are exposed and accessible to the host's immune system, outer membrane proteins (OMP's) make good vaccine candidates. Therefore, in a preferred embodiment, said immunogenic compound is an outer membrane fragment, or outer membrane vesicle. The preparation of outer membranes of bacteria is known in the art. U.S. Pat. No. 4,707,543 describes an outer membrane complex which is isolated from the bacterium. Proc. Int. Pig Veter. Soc. 10 81 (1988) describes an outer membrane vaccine for pigs derived from Haemophilus pleuropneumoniae (now known as Actinobacillus pleuropneumoniae). The vaccine contained APP outer membranes. Bacterial outer membranes may be produced by sonication of lysozyme-sucrose treated cells and then sucrose density gradient centrifugation. The sonication is preferably performed for 10-15 seconds. Lysozyme degrades peptidoglycan (cell wall). Sucrose maintains the cell membranes remaining after treatment with the lysozyme until the cells are sonicated.

In an embodiment, said immunogenic compound comprises a protein derived from the outer membrane of Ralstonia Pickettii or a fragment or a variant of said protein, wherein the protein, fragment, or variant is capable of producing a protective immune response in a subject. Several proteins are known which reside within the outer membrane of Ralstonia Pickettii, including but not limited to BamB (Refseq ID YP_002981118.1. and NC_012856.1), Rpic12D_5148 outer membrane efflux protein (Refseq ID NC_012849.1), Rpic12D_5135 outer membrane efflux protein (Refseq ID NC_012849.1), Rpic12D_5289 outer membrane efflux protein (Refseq ID NC_012849.1), Rpic 4684 outer membrane efflux protein (Refseq ID NC_010678.1), Rpic12D 4293 (RefSeq-Pro:YP_002984208.1), Rpic12D_0604 (RefSeq-Pro:YP_002980581.1), Rpic_3640 (RefSeq-Pro:YP_001901191.1). Also, recombinant membrane proteins of Ralstonia Pickettii are known, for instance Outer-membrane lipoprotein lolB (lolB) catalogue No. MBS1169212 are available from http://www.mybiosource.com/.

As used herein the terms “outer layer protein”, “surface layer protein” and “outer membrane protein” may be used interchangeably and mean a protein which is present, partially or completely, on a bacterial cell surface and includes proteins which are permanently or generally located on the cell surface and also proteins which are exported from the bacterium to the cell surface occasionally, for example when the cell is under stress, or temporarily, for example during a particular phase of the fife cycle of the cell.

As used herein the term “fragment” in the context of a protein, refers to any portion of the given amino acid sequence of a polypeptide or protein which has the same activity as the complete amino acid sequence. Fragments will suitably comprise at least 5 and preferably at least 10 consecutive amino acids from the basic sequence and does include combinations of such fragments. In order to retain activity, fragments will suitably comprise at least one epitopic region. Fragments comprising epitopic regions may be fused together to form a variant.

In the context of the present invention the expression “variant” as used herein refers to sequences of amino acids which differ from the base sequence from which they are derived in that one or more amino acids within the sequence are substituted for other amino acids. Amino acid substitutions may be regarded as “conservative” where an amino is replaced with a different amino acid with broadly similar properties. “Non-conservative” substitutions are where amino acids are replaced with amino acids of a different type. Broadly speaking, fewer non-conservative substitutions will be possible without altering the biological activity of the polypeptide. Suitably variants will be greater than 75% identical, preferably at least 80% identical, more preferably at least 85% identical, and most preferably at least 90% identical to the base sequence. Variants included in the description of the present invention are intended to exclude substitutions which result in the variant having a substantially identical sequence to a genomic sequence from another organism.

In another embodiment, said immunogenic compound comprises Ralstonia pickettii polysaccharides such as capsular polysaccharides or lipopolysaccharides, optionally in combination with further Ralstonia pickettii-specific antigens such as those mentioned above. In an embodiment, said immunogenic compound comprises an Ig-like molecule such as an antibody, or a binding fragment thereof which is capable of neutralizing Ralstonia pickettii and/or binds specifically to a Ralstonia pickettii cell or a part thereof.

The term “antibody” as used herein is well-known in the art. It refers to any polypeptide comprising an antigen-binding site with at least one complementarity determining region (CDR). The term includes, but is not limited to, polyclonal antibodies, monoclonal antibodies, monospecific antibodies, multispecific antibodies, humanized antibodies, chimeric antibodies, human antibodies, and single-chain antibodies (e.g., VHH). In the context of the present invention, the term “antibody” also includes antibody fragments such Fab, F(ab′)2, Fv, scFv, Fd, dAb, and other antibody fragments or other constructs comprising CDRs that retain antigen-binding function. Typically, such fragments would comprise an antigen-binding domain. The antibodies may be any of the known antibody isotypes and their conformations, for example, IgA, such as IgA1 or IgA2, IgD, IgE, IgG, such as IgG1, IgG2a, IgG2b, IgG3, IgG4, or IgM class, or may constitute mixtures thereof in any combination, such as a mixture of antibodies from the IgG1 and IgG2a class.

Preferred antibodies are capable of neutralizing Ralstonia pickettii cells. Said antibodies or binding fragments thereof may be polyclonal or monoclonal, and may be produced using conventional methods. For instance, polyclonal antibodies may be generated by immunisation of an animal (such as a rabbit, rat, goat, horse, sheep etc.) with immunogenic proteins or immunogenic subunits or fragments thereof, to raise antisera, from which antibodies may be purified. Monoclonal antibodies may be obtained by fusing spleen cells from an immunised animal with myeloma cells, and selecting hybridoma cells which secrete suitable antibodies, preferably as described in Nat Med. 2010 January; 16(1):123-8.

In the present invention, the term “binding fragment” is understood as a part or portion of an Ig-like molecule, e.g., antibody, as taught herein, which is capable of neutralizing Ralstonia pickettii and/or specifically binds to Ralstonia pickettii. Binding fragments (also referred to as “antigen-binding fragment”) may be obtained via chemical or enzymatic treatment of an intact or complete Ig-like molecule, e.g., antibody. Alternatively, binding fragments may be obtained using standard molecular biology techniques and protocols. Non-limiting examples of binding fragments of Ig-like molecules include Fab, Fab′, F(ab′)2, and Fv fragments; diabodies; linear antibodies and single-chain antibody molecules.

In addition, the use of bacteriophages to specifically eliminate bacterial contaminations has shown to be effective (Dalmasso M., et al. Trends Microbiol. 2014 July; 22(7):399-405). Biocontrol of Ralstonia cells by treatment with lytic bacteriophages has been described in Fujisawa et al., Appl Environ Microbiol. 2011 June; 77(12):4155-62. Therefore, in another preferred embodiment, said compound comprises a bacteriophage which is effective against Ralstonia pickettii. Ralstonia pickettii specific bacteriophages are known in the art, for instance Ralstonia pickettii bacteriophage p12J, which has been sequenced (GenBank accession number NP_932302).

Pharmaceutical Compositions

In a further aspect, the invention provides a pharmaceutical composition for use in the treatment or prevention of insulin resistance, obesity or type II diabetes of a subject, comprising the compound effective against Ralstonia pickettii as defined above and a pharmaceutically-acceptable excipient, carrier or diluent.

In one embodiment, the compound of the invention is administered in a pharmaceutically-acceptable carrier. Any suitable carrier known in the art may be used. Carriers that efficiently solubilize the compound are preferred. Carriers include, but are not limited to, a solid, liquid, or a mixture of a solid and a liquid. The carriers may take the form of capsules, tablets, pills, powders, lozenges, suspensions, emulsions, or syrups. The carriers may include substances that act as flavouring agents, lubricants, solubilizers, suspending agents, binders, stabilizers, tablet disintegrating agents, and encapsulating materials.

Non-limiting examples of materials which can serve as pharmaceutically-acceptable carriers include: (1) sugars, such as lactose, glucose, and sucrose; (2) starches, such as corn starch and potato starch; (3) cellulose and its derivatives, such as sodium carboxymethyl cellulose, ethyl cellulose, and cellulose acetate; (4) powdered tragacanth; (5) malt; (6) gelatine; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerine, sorbitol, manitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline, (18) Ringer's solution, (19) ethyl alcohol; (20) phosphate buffer solutions; and (21) other non-toxic compatible substances employed in pharmaceutical compositions.

The compositions of the invention may conveniently be presented in unit dosage form and may be prepared by any methods well known in the art of pharmacy. The amount of active ingredient which can be combined with a carrier material to produce a single-dosage form will vary depending upon the subject being treated, the particular mode of administration, and the particular condition being treated, among others. The amount of active ingredient that can be combined with a carrier material to produce a single-dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these compositions include the step of bringing into association the compound of the invention with the carrier and, optionally, one or more accessory ingredients. In general, the compositions are prepared by uniformly and intimately bringing into association an antibiotic compound of the present invention with liquid carriers, or timely divided solid carriers, or both, and then, if necessary, shaping the product.

In solid dosage forms of the invention for oral administration (e.g., capsules, tablets, pills, dragees, powders, granules, and the like), the active ingredient is mixed with one or more additional ingredients, such as sodium citrate or dicalcium phosphate, and/or any of the following: fillers or extenders, such as, but not limited to, starches, lactose, sucrose, glucose, manitol, and/or silicic acid; binders, such as, but not limited to, carboxymethylcellulose, alginates, gelatine, polyvinyl pyrrolidone, sucrose, and/or acacia; humectants, such as, but not limited to, glycerol; disintegrating agents, such as, but not limited to, agar, calcium carbonate, potato or tapioca starch, alginic acid, certain silicates, and sodium carbonate; solution retarding agents, such as, but not limited to, paraffin; absorption accelerators, such as, but not limited to, quaternary ammonium compounds; wetting agents, such as, but not limited to, cetyl alcohol and glycerol monostearate; absorbents, such as, but not limited to, kaolin and bentonite clay; lubricants, such as, but not limited to, talc, calcium stearate, magnesium stearate, solid polyethylene glycols, sodium lauryl sulphate, and mixtures thereof; and colouring agents. In the case of capsules, tablets, and pills, the pharmaceutical compositions may also comprise buffering agents. Solid compositions of a similar type may also be employed as fillers in soft and hard-filled gelatine capsules using such excipients as lactose or milk sugars, as well as high molecular weight polyethylene glycols, and the like.

In powders, the carrier is a finely-divided solid, which is mixed with an effective amount of a finely-divided agent. Powders and sprays can contain, in addition to a compound of this invention, excipients, such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Tablets for systemic oral administration may include one or more excipients as known in the art, such as, for example, calcium carbonate, sodium carbonate, sugars (e.g., lactose, sucrose, manitol, sorbitol), celluloses (e.g., methyl cellulose, sodium carboxymethyl cellulose), gums (e.g., arabic, tragacanth), together with one or more disintegrating agents (e.g., maize, starch, or alginic acid, binding agents, such as, for example, gelatine, collagen, or acacia), lubricating agents (e.g., magnesium stearate, stearic acid, or talc), inert diluents, preservatives, disintegrants (e.g., sodium starch glycolate), surface-active and/or dispersing agent. A tablet may be made by compression or moulding, optionally with one or more accessory ingredients.

In solutions, suspensions, emulsions or syrups, an effective amount of the antibiotic compound is dissolved or suspended in a carrier, such as sterile water or an organic solvent, such as aqueous propylene glycol. Other compositions can be made by dispersing the agent in an aqueous starch or sodium carboxymethyl cellulose solution or a suitable oil known to the art. The liquid dosage forms may contain inert diluents commonly used in the art, such as, for example, water or other solvents, solubilizing agents and emulsifiers, such as, but not limited to, ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan, and mixtures thereof.

Besides inert diluents, the oral compositions can also include adjuvants, such as wetting agents, emulsifying and suspending agents, sweetening, flavouring, colouring, perfuming, and preservative agents.

Suspensions, in addition to the active compound, may contain suspending agents as, for example, ethoxylated isostearyl alcohols, polyoxyethylene sorbitol and sorbitan esters, microcrystalline cellulose, aluminum metahydroxide, bentonite, agar and tragacanth, and mixtures thereof.

Pharmaceutical compositions for rectal or vaginal administration may be presented as a suppository, which may be prepared by mixing one or more compounds of the invention with one or more suitable non-irritating excipients or carriers comprising, for example, cocoa butter, polyethylene glycol, a suppository wax or a salicylate, and which is solid at room temperature but liquid at body temperature and, thus, will melt in the rectum or vaginal cavity and release the agents. Compositions suitable for vaginal administration also include pessaries, tampons, creams, gels, pastes, foams, or spray compositions containing such carriers as are known in the art to be appropriate.

Dosage forms for the topical or transdermal administration of the compound of this invention include powders, sprays, ointments, pastes, creams, lotions, gels, solutions, patches, and inhalants. The active antibiotic compound may be mixed under sterile conditions with a pharmaceutically-acceptable carrier, and with any preservatives, buffers, or propellants that may be required.

Ointments, pastes, creams, and gels may contain, in addition to the active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of a compound of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the antibiotic compound in a polymer matrix or gel.

Active Vaccines

In a preferred embodiment, said composition of the invention is a vaccine comprising an immunogenic compound as taught herein. Said immunogenic compound is in certain embodiments part of a Ralstonia pickettii bacterium or a fragment thereof.

The invention further provides a vaccine composition comprises a Ralstonia pickettii bacterium which is live, inactivated, attenuated or a fragment thereof capable of producing a protective immune response in an animal. Preferred are vaccines comprising inactivated whole bacteria. In another embodiment, said vaccine comprises a fragment of said Ralstonia pickettii bacterium which is capable of producing a protective immune response in an animal. Said vaccine is suitable for use in any treatment wherein Ralstonia pickettii is a causative agent. Preferably, said vaccine composition is for use in the treatment or prevention of insulin resistance, obesity, type II diabetes, metabolic syndrome, NAFLD or NASH in a subject.

Vaccine compositions of the invention may be administered in a suitable, nontoxic pharmaceutical carrier, may be comprised in microcapsules, and/or may be comprised in a controlled release, e.g., sustained release, implant. Said vaccine of the invention may be used in conjunction with other bactericidal or bacteriostatic methods, preferably with a compound or composition as taught herein.

Suitable excipients and carriers, which may be used in the vaccine composition will be known to those skilled in the art. These may include solid or liquid carriers. Suitable liquid carriers include water or saline. The polypeptides and proteins of the composition may be formulated into an emulsion or alternatively they may be formulated in, or together with, biodegradable microspheres or liposomes. Suitably, the vaccine composition further comprises an adjuvant which stimulates the host's immune response. Particularly suitable adjuvants include Alhydrogel, MPL+TDM and Freunds Incomplete Adjuvant. Most preferred is Freunds Incomplete Adjuvant.

In addition to inactivated, attenuated or live bacterial isolates, said vaccine composition of the invention can also include an appropriate amount of one or more commonly used adjuvants. Suitable adjuvants may include, but are not limited to: mineral gels, e.g., aluminum hydroxide; surface active substances such as lysolecithin; glycosides, e.g., saponin and saponin derivatives such as Quil A or GPI-0100; cationic surfactants, e.g. DDA (quaternary hydrocarbon ammonium halogenides, pluronic polyols; polyanions and polyatomic ions; polyacrylic acids, non-ionic block polymers, e.g., Pluronic F-127 (B.A.S.F., USA); Avridine and Rantidine; peptides; recombinant mutant labile toxins, e.g., leukotoxin (LT) or cholera toxin (CT); chemically bound or close proximity molecular transporters; mineral oils, e.g. Montanide ISA-50 (Seppic, Paris, France), carbopol, Amphigen (Hydronics, USA), Omaha, Neb. USA, Alhydrogel, (Superfos Biosector, Frederikssund, Denmark) oil emulsions, e.g. an emulsion of mineral oil such as BayolF/Arlacel A and water, or an emulsion of vegetable oil, water and an emulsifier such as lecithin; alum, cholesterol cytokines and combinations of adjuvants. Polyatomic ions can also function as dispersing, thickening and anticaking agents, which allow the vaccine to be resuspended as a monodisperse suspension after a prolonged period of settling. The adjuvant combinations may be presented in aqueous, encapsulated (controlled or delayed release) or microencapsulated forms.

Vaccines of the present invention may be administered locally and/or systemically by any method known in the art, including, but not limited to, intravenous, subcutaneous, intramuscular, intravaginal, intraperitoneal, intranasal, oral or other mucosal routes. Said vaccine may desirably be administered at several intervals in order to sustain antibody levels.

### Antibodies

In another preferred embodiment of the composition, said composition comprises an antibody which is capable of neutralizing Ralstonia pickettii and/or binds specifically to Ralstonia pickettii, or a binding fragment thereof and a pharmaceutically-acceptable excipient, carrier, or diluent. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulphite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatine. Sterile injectable solutions can be prepared by incorporating the antibody according to the invention in the required amount in an appropriate solvent with one or a combination of ingredients enumerated above, as required, followed by filtered sterilization.

Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the composition. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811. It is especially advantageous to formulate oral or parenteral compositions in dosage unit form for ease of administration and uniformity of dosage.

It will be appreciated that treatment as described herein includes preventing a disease, ameliorating symptoms, slowing disease progression, reversing damage, or curing a disease.

All compositions of the invention can be included in a container, pack, kit, or dispenser together with instructions for administration.

### Methods of Diagnosis and Prediction

The inventors have found that bacteria of the gram negative bacterial strain Ralstonia pickettii were present in an increased level in test samples of patients suffering from obesity, both in faeces and mesenteric visceral adipose tissue. In addition, the inventors have shown that the concentrations of Ralstonia pickettii correlated with both clinical and epidemiological features of insulin resistance. The invention therefore provides a method of diagnosis or prediction of insulin resistance, obesity, type II diabetes, metabolic syndrome, NAFLD or NASH in a test sample of a subject. The method helps identifying a group of subjects which may benefit from a treatment as described herein.

The methods described herein may include identifying a subject in need of treatment. In a preferred embodiment, the methods include identifying a mammal in need of treatment. In a highly preferred embodiment, the methods include identifying a human in need of treatment. Identifying a subject in need of treatment may be accomplished by any means that indicates a subject who may benefit from treatment. The methods may include identifying a subject at risk for or suffering from insulin resistance, obesity, type II diabetes, metabolic syndrome, NAFLD or NASH or a condition associated with these diseases and administering a compound to the subject in an effective amount to treat or prevent the condition. For example, identifying a subject in need of treatment may occur by clinical diagnosis, laboratory testing, or any other means known to one of skill in the art, including any combination of means for identification. In a preferred embodiment, said method further comprises the determination of the level of adiponectin in a test sample of said subject, preferably a plasma sample. A low level of adiponectin is preferably used to predict an increased risk of developing insulin resistance, obesity, type II diabetes, metabolic syndrome, NAFLD or NASH. In another preferred embodiment, the amount of leukocytes is also determined in a test sample of the subject. The amount of leukocytes is also indicative of said diseases.

Preferably, said test sample according to the present invention is a blood, serum, plasma, leucocytes, stool, adipose tissue or hepatic tissue sample. Most preferred test samples include stool and visceral adipose tissue samples. Highly preferred samples include faeces and mesenteric visceral adipose tissue samples.

The method of the invention comprises determining the level of Ralstonia pickettii. The presence of said bacterium may be determined by any method known in the art. Any compound, molecule or cell fragment of Ralstonia pickettii which enables the identification thereof may be used as a biomarker for its level. Suitable methods for detecting bacteria in a test sample include the detection of a nucleic acid or a protein of Ralstonia pickettii. The genome of Ralstonia pickettii is known in the art. Therefore, a skilled person in the art can readily and without undue experimentation design nucleic acids which hybridize under stringent conditions to a Ralstonia pickettii specific nucleic acid. For example, primers may be designed which enable the amplification of nucleic acids which help identifying Ralstonia pickettii. Such techniques are well known in the art and described herein. Preferably, said nucleic acid is species specific. Species specific nucleic acids for Ralstonia pickettii are disclosed in Ryan et al., BMC Microbiology 2011, 11:194.

Preferably, said method comprises comparing said level with a reference value or the level of Ralstonia pickettii or the presence of an antibody which specifically binds to Ralstonia pickettii in a control sample. Said control sample may be a positive control sample or a negative control sample. Said reference value may be based on a predetermined level in a control sample. Diagnosing or predicting said disease is based on said comparison. The level of Ralstonia pickettii or the level of an antibody which specifically binds to Ralstonia pickettii in said test sample is indicative or predictive of the disease of the invention.

In a preferred embodiment, the method of the invention comprises the detection of 16S DNA of Ralstonia pickettii. In a preferred embodiment, the method of the invention comprises the detection of 16S rRNA or 16S rDNA (the gene coding for the 16S rRNA) of Ralstonia pickettii. Preferably, species-specific PCR primers are used to amplify the 16S DNA of Ralstonia pickettii. Preferably, said primers include forward primer having the nucleic acid sequence ATGATCTAGCTTGCTAGATTGAT (SEQ ID NO: 1) and reverse primer having the nucleic acid sequence ACTGATCGTCGCCTTGGTG (SEQ ID NO: 2).

Someone skilled in the art will find that it will be useful to develop also other primers will be useful targeting the 16S rRNA of Ralstonia pickettii strains as deposited in the SILVA database (http://www.arb-silva.de/documentation/release-119/). Similarly, primers for one of the genomes of Ralstonia pickettii can be used to develop specific detection methods. Preferably, genomic DNA of both prokaryotic and eukaryotic origin is isolated from visceral adipose tissue according to Zoetendal et al. (16). Briefly, tissues are first treated with a mix of SDS and proteinase K at 55° C. and homogenized by mechanical disruption in the presence of phenol and zirconia glass beads (1 mm) in the FAST Prep-24 (MP Biomedical). The released genomic DNA may subsequently be further extracted with a number of phenol/chloroform extractions and precipitated in the presence of absolute ethanol.

Preferably, species specific PCR primers are used to amplify the 16S DNA of Ralstonia pickettii. Preferably, said primers include forward primer having the nucleic acid sequence ATGATCTAGCTTGCTAGATTGAT (SEQ ID NO: 1) and reverse primer having the nucleic acid sequence ACTGATCGTCGCCTTGGTG (SEQ ID NO: 2). In another embodiment, universal 16S primers are used to amplify any 16S nucleic acid and use nested primers which are specific for Ralstonia pickettii 16S to further amplify Ralstonia pickettii 16S nucleic acids. In the context of the invention, the term “universal primers” refers to primers comprising a sequence which is able to hybridize to 16S nucleic acids from essentially any origin. Preferably, the universal primers according to the invention are primers comprising a sequence selected from the group consisting of the sequence 5′-GTTTGATCCTGGCTCAG-3′ (SEQ ID NO: 3) and the sequence 5′-GCCCGGGAACGTATTCACCG-3′ (SEQ ID NO: 4). In said embodiment, the following primers are preferably used: Forward (nested): CGCCCGGGGCGCGCCCCGGGCGGGGCGGGG (SEQ ID NO: 5) and Reverse (nested): GCACGGGGGGAACGCGAAGAACCTTAC (SEQ ID NO: 6). The amplified products may be analysed using any method known in the art to identify whether they are derived from Ralstonia pickettii. For instance, by sequencing said amplified products and subsequently determine whether the nucleic acid sequence is a sequence from Ralstonia pickettii by comparing the sequence data from the fragments with the known sequence data of Ralstonia pickettii. Preferably, a quantitative PCR method is used to determine the amount of a nucleic acid from Ralstonia pickettii.

Level of Ralstonia in faecal samples may suitably be determined following isolation of bacterial cDNA as previously described (17) and total bacteria qPCR can be performed as described (21). Ralstonia pickettii specific DNA amplification may be performed as described herein.

In another embodiment of the method of the invention, the presence of Ralstonia pickettii in a test sample is determined using specific antibodies which bind to this bacterium. Methods of producing antibodies which bind specifically to Ralstonia pickettii or a fragment thereof which is suitable for identification, are well-known in the art. Suitable antibodies can readily be generated by the skilled person.

Such a method comprises contacting the test sample suspected of containing Ralstonia pickettii cells with an antibody raised against any of the proteins as hereinbefore described, or a binding fragment of said antibody, and detecting binding there between.

Detection methods used include conventional immunological methods for example ELISA, surface plasmon resonance and the like.

Suitably the antibody or binding fragment is immobilised on a solid support, for example on an ELISA plate, but other forms of support, for example membranes such as those utilised in conventional “dip-stick” tests may also be employed.

Detection of a complex between a surface layer protein within in the sample, and a binding moiety as described above can be detected using conventional methods, in particular immunological methods such as ELISA methods. Assay formats may take various forms including “sandwich” or “competitive” types.

In a typical sandwich assay, the binding moiety is immobilised on a support, such as an ELISA plate, where is it contacted with said test sample suspected of containing Ralstonia pickettii cells. Where present, these cells will bind the binding moiety and so become immobilised in their turn. The support is then separated from the sample, for example by washing. The presence of the cells on the support can then be detected by application of secondary antibodies or binding fragments thereof, which bind to the cells, and are detectable, for example because they are labelled for instance with a visible label such as a fluorescent label, or a radiolabel, but preferably that they can be developed to produce a visible signal. A particular example of a secondary antibody is an antibody, or binding fragment, that carries an enzymatic label, such as horseradish peroxidase, which can then be utilised to produce a signal by addition of the enzyme substrate, using conventional ELISA methodology.

In a particular competitive assay format, the binding moiety of the invention is immobilised on a support. In this instance, a protein which binds said binding moiety in competition to the cells is added to the sample prior to contact with the support. Any cells present within the sample will compete with this protein for binding to the immobilised binding moiety. Thus, the absence of peptide on the support is indicative of the presence of cells in the sample.

In this case, the competing protein is suitably labelled so that it may be readily detected, for instance using a visible label such as a fluorescent or radiolabel. Alternatively, it may be detected using a secondary antibody or a binding fragment thereof, such as those discussed above in relation to sandwich assays, which binds the protein.

In another embodiment of the method of the invention, the presence of Ralstonia pickettii is detected indirectly, for instance by determining the presence of an antibody capable of specifically binding to Ralstonia pickettii in a test sample of said subject. Methods of determining a titer against known antigens are known in the art. The antibodies induced by the subject can be harvested and isolated to the extent desired by well-known techniques, such as by alcohol fractionation and column chromatography, or by immunoaffinity chromatography; that is, by binding antigen to a chromatographic column packing like Sephadex®, passing the antiserum through the column, thereby retaining specific antibodies and separating out other immunoglobulins (IgGs) and contaminants, and then recovering purified antibodies by elution with a chaotropic agent, optionally followed by further purification, for example, by passage through a column of bound blood group antigens or other non-pathogen species. Said antigens may be from fractionated Ralstonia pickettii cells or any immunogenic compound of the invention as described herein. This procedure may be preferred when isolating the desired antibodies from the sera or plasma of subjects that have developed an antibody titter against Ralstonia pickettii, thus assuring the retention of antibodies that are capable of binding to the antigen.

Methods of the invention for the detection of antibodies or said bacteria in a host may include immunoassays. Such immunoassays are known in the art and include, but are not limited to radioimmunoassays (RIA), enzyme-linked immunosorbent assays (ELISA), fluorescent immunoassays, and fluorescence polarization immunoassays (FPIA).

In some embodiments, the method comprises a step of culturing Ralstonia pickettii bacteria to increase the number of bacterial cells.

An increased level of Ralstonia pickettii in said test sample of the subject, is indicative of an increased chance that the subject is suffering from or will develop insulin resistance, obesity, type II diabetes, metabolic syndrome, NAFLD or NASH. Preferably, patients who have been identified as carrying increased levels of Ralstonia pickettii compared to a control sample derived from healthy subjects may be treated using any of the compounds or compositions taught herein.

The method of the invention can also be utilized in the assessment of the severity of the disease and appropriate methods of treatment. Assays for the quantity (absolute or relative) of Ralstonia pickettii may be carried out and the results interpreted in a variety of ways, depending on the assay format, the nature of the sample being assayed, and the information sought.

The quantity of Ralstonia pickettii bacteria in the test sample may be used to determine the severity of the disease status. In a preferred embodiment, the amount of Ralstonia pickettii bacterial load is determined in a faeces sample. The inventors have shown that increased Ralstonia pickettii bacterial load in said test sample of the subjects is highly predictive for the development of insulin resistance.

Quantitative measurement can be achieved by a variety of methods known in the art. These methods include, but are not limited to: antibody/homologous cross reactive but specific reagents, quantitative polymerase chain reaction, and any “sandwich” assay—both antibody and nucleic acid based. Further included are direct antigen measurement and lateral flow immunoassay including utilizing surface enhanced Raman scattering (SERS) technology.

Use of Ralstonia pickettii Specific Antibodies, Nucleic Acids, Kits

In a further aspect, the invention provides the use of an antibody binding specifically to an antigen of Ralstonia pickettii, a Ralstonia pickettii cell or an immunogenic part thereof, and/or a nucleic acid hybridizing under stringent conditions to a nucleic acid from Ralstonia pickettii in a method of diagnosis or prediction of insulin resistance, obesity, type II diabetes, metabolic syndrome, NAFLD or NASH as taught herein.

In another aspect the invention provides a diagnostic kit comprising one or more of said antibody binding specifically to an antigen of Ralstonia pickettii, Ralstonia pickettii cell or an immunogenic part thereof, and/or nucleic acid hybridizing under stringent conditions to nucleic acid from Ralstonia pickettii as described above in a method of diagnosis or prediction of insulin resistance, obesity, type II diabetes, metabolic syndrome, NAFLD or NASH as taught herein.

In a preferred embodiment, said nucleic acid hybridizing under stringent conditions is a probe or a primer. The primer is preferably single stranded for maximum efficiency in amplification. Preferably, the primer is an oligodeoxyribonucleotide. The length of the primer depends on several factors, including temperature and sequence of the primer, but must be long enough to initiate the synthesis of amplification products. Preferably the primer is from 10 to 35 nucleotides in length. More preferably, the primer is from 15 to 30 nucleotides in length. Most preferably, the primer is 22 or 29 nucleotides in length. A primer can further contain additional features which allow for detection, immobilization, or manipulation of the amplified product. The primer may furthermore comprise covalently-bound fluorescent dyes, which confer specific fluorescence properties to the hybrid consisting of the primer and the target sequence or non-covalently bound fluorescent dyes which can interact with the double-stranded DNA/RNA to change the fluorescence properties. Fluorescent dyes which can be used are for example SYBR-green or ethidium bromide.

In an embodiment, said kit comprises all of the essential reagents required to perform said diagnostic or prediction method according to the present invention. The kit may be presented in a commercially packaged form as a combination of one or more containers holding the necessary reagents. Such a kit comprises a monoclonal or polyclonal antibody in combination with several conventional kit components. The kit may further comprise an Ralstonia pickettii bacteria as a control sample. Conventional kit components will be readily apparent to those skilled in the art and are disclosed in numerous publications, including Antibodies A Laboratory Manual (E. Harlow, D. Lane, Cold Spring Harbor Laboratory Press, 1989). Conventional kit components may include such items as, for example, microtiter plates, buffers to maintain the pH of the assay mixture (such as, but not limited to Tris, HEPES, etc.), conjugated second antibodies, such as peroxidase conjugated anti-mouse IgG (or any anti-IgG to the animal from which the first antibody was derived) and the like, and other standard reagents.

It is contemplated that other Ralstonia species than Ralstonia pickettii, especially of the species Ralstonia mannitolilytica and Ralstonia eutropha may also be implicated in insulin resistance, obesity, type II diabetes, metabolic syndrome, NAFLD or NASH. A skilled person in the art will therefore understand that in cases herein where reference is made to “Ralstonia pickettii”, this term may in aspects of the invention be understood to refer to other Ralstonia species as well, and in particular to Ralstonia mannitolilytica and Ralstonia eutropha. Therefore, compounds or compositions which are effective against other Ralstonia species, method of diagnosis or prediction of insulin resistance, obesity or type II diabetes in a subject comprising determining the presence of Ralstonia pickettii or the presence of an antibody which specifically binds to other species of Ralstonia in a test sample of a subject, and the use of compounds or a kit for the detection of other species of Ralstonia and are also within the scope of this invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### Examples

### Participants

Caucasian subjects (male/postmenopausal females, scheduled for elective laparoscopic cholecystectomy) were screened by the attending surgeon. Inclusion criteria were age between 18-75 years and body-mass index (BMI) between 25-40 kg/m². Exclusion criteria were malignancy, known T2D, generalized inflammation and use of probiotics and/or antibiotics in the past three months. Written informed consent was obtained from all subjects. The study was approved by AMC Ethics committee and conducted at the Flevo hospital (Almere), Sint Lucas Andreas hospital (Amsterdam) and Academic Medical Center (Amsterdam), in accordance with the Declaration of Helsinki. Participants could continue their own diet, but were asked to fill out a week-long online nutritional diary (www.dieetinzicht.nl) to monitor caloric intake. Prior to surgery, anthropometric measurements and blood sampling was performed for fasting plasma levels of metabolic parameters. Total cholesterol, low density lipoprotein cholesterol (LDLc), high density lipoprotein cholesterol (HDLc) and triglycerides (TG) were measured by using commercially available enzymatic assays (Randox, USA and Wako, USA). All analyses were performed using a Cobas Mira autoanalyzer (Horiba, France). C-Reactive Protein (CRP, Roche Diagnostics) and total adiponectin (RIA from Linco and for DIWA study ELISA by Abcam) were determined. Insulin resistance was calculated using the homeostasis model assessment (HOMA-IR) (see Table 1). Finally, a fresh morning stool sample was stored at −80° C. until DNA was extracted. During the sterile laparoscopic surgical procedure mesenteric-visceral (colon transversum), omental (right upper quadrant abdomen) and subcutaneous fat (subxyphoidal laparoscopic entrance) biopsies were taken at same location in all patients, directly collected in sterile, RNase-, DNase- and pyrogen free microtubes (Eppendorf), promptly frozen in liquid nitrogen and transferred to a −80° C. freezer for later analysis. Fecal DNA was extracted using standard methods (8).

For the DIWA study, 70-year-old women were included if they had type 2 diabetes mellitus (T2D), impaired glucose tolerance (IGT) or normal glucose tolerance (NGT) described elsewhere (8). The Exclusion criteria were chronic inflammatory disease and treatment with antibiotics during the preceding 3 months. All subjects gave informed consent and provided a fresh morning stool sample.

TABLE 1
Baseline Characteristics of study subjects

| (N = 12) | | |
|---|---|---|
| Male/Female | 7/5 | |
| Age (years) | 41 | (35-46) |
| Body-mass index (kg/m²) | 26.0 | (23-37) |
| Homeostatic Model Assessment | 4.7 | (2.4-9.0) |
| Cholesterol (mmol/liter) | 5.1 | (4.5-5.5) |
| HDLc | 1.2 | (0.9-1.5) |
| LDLc | 3.2 | (2.5-3.5) |
| TG | 1.6 | (1.0-7.4) |
| CRP (mg/L) | 2.6 | (1.0-7.4) |
| Caloric intake (kcal/day) | 1815 | (1585-3190) |

Data are presented as median (range)

### Bacterial DNA Isolation and Sequencing

Genomic DNA of both prokaryotic and eukaryotic origin was isolated from visceral adipose tissue according to Zoetendal et al. (16). Briefly, tissues were first treated with a mix of SDS and proteinase K at 55° C. and homogenized by mechanical disruption in the presence of phenol and zirconia glass beads (1 mm) in the FAST Prep-24 (MP Biomedical). The released genomic DNA was further extracted with a number of phenol/chloroform extractions and precipitated in the presence of absolute ethanol. The prokaryotic fraction was studied by using a range of 16S rRNA gene specific primers and assays. From the minute amounts of bacterial genomic DNA, full-length16S rDNA amplicons were generated in a PCR by using primers Bact-27F (5′GTTTGATCCTGGCTCAG-3′) and Prok-1392R (5′GCCCGGGAACGTATTCACCG-3′) using PCR conditions described by Rajilic-Stojanovic et al (17). The resulting amplicons were used as input for a nested PCR using primers 968-GC-F (5′CGCCCGGGGCGCGCCCCGGGCGGGGCGGGG-GCACGGGGGGAACGCGAAGAACCTTAC) and 1392R (18), generating fragments fit for a diversity analysis by DGGE (denaturing gene gel electrophoresis) using conditions described by Heilig et al. (19). The dominant band appearing in the DGGE analyses was identified by cloning the DGGE amplicon in a pGEM-T easy vector (Promega, Leiden, The Netherlands) and transforming them to Stratagene E. coli XL-1 Blue competent cells (Agilent Technologies, Amstelveen, The Netherlands) according to the manufacturers' specifications. Clones containing the right size insert and migrating to the same position as the dominant band in the DGGE gel were subjected to Sanger sequence analysis (GATC Biotech, Konstanz, Germany). Sequences were identified by performing a BLAST search (http://blast.ncbi.nlm.nih.gov/Blast.cgi). Sequence analysis of the dominant band appearing in DGGE showed highest similarity to sequences of Ralstonia species. To estimate the Ralstonia-like bacteria within the total bacterial fraction, two nested quantitative PCR assays were performed on the previously generated 16S rDNA amplicons. Ralstonia being a member of the group of Burkholderiales comprising of 4 different bacterial subgroups, was detected (20).

Ralstonia spp. were quantified in faecal samples (from both above mentioned subjects as well as from subjects from the DIWA study (8), and total bacteria qPCR was performed (21). Used primers for faecal Ralstonia-specific detection were as follows: Ralstonia pickettii: Forward:ATGATCTAGCTTGCTAGATTGAT Reverse: ACTGATCGTCGCCTTGGTG. Ralstonia. mannitolitytica: Forward GGGAAAGCTTGCTTTCCTGCC Reverse: TCCGGGTATTAACCAGAGCCAT. Ralstonia insidiosa: Forward ATGATCTAGCTTGCTAGATTGAT Reverse CACACCTAATATTAGTAAGTGCG. Ralstonia eutropha: Forward ACCCCGGGGTCGATGACGGTA Reverse GCCTTGCAGTCACAAGCGCC) (22).

### Ralstonia Pickettii Inoculation Experiments in DIO Mice

To demonstrate causality and to exclude the possibility of contamination, we performed inoculation studies in male C56BL6/J mice fed a 60% high fat diet (Harlan Tek) at 4 weeks of age to induce diet induced obesity (DIO). Mice were in a constant 12-hour light-dark cycle with controlled temperature and humidity and were given access to food (high fat diet) and water ad libitum. Body weight and food intake were measured once a week. Starting at the age of 12 weeks, daily intragastrical gavage of 10⁶ CFU Ralstonia pickettii (DSM 6297) in 100 μl glycerol vehicle (20 mM) versus heat inactivated Ralstonia pickettii (DSM 6297) as a first and 10% glycerol vehiculum as a second control (n=10 mice per group) was given for 4 weeks. At week 17, oral glucose tolerance test (2 g glucose/kg bodyweight) was carried out followed by blood sampling every 30 minutes for 2 hours. Blood was collected via tail bleeding at t=0, 10, 20, 30, 60, 90, and 120 minutes. Animals were sacrificed by sodium pentobarbital and cervical dislocation. Plasma was collected by cardiac puncture for later analyses including glucose/insulin levels. HOMA-IR was defined as [fasting plasma insulin (μU/mL)*fasting plasma glucose (mmol/L)]/22.5. Faeces was collected from the cecum and colon for later analyses of Ralstonia concentrations (22). Also, adipose tissue specimen of visceral mesenteric (small intestine), kidney and epididymal adipose tissue was collected, weighed, snap frozen in liquid nitrogen and stored at −80C for later analyses including TLR gene expression.

Preparation of Ralstonia pickettii Vaccine

Cells of Ralstonia pickettii were centrifuged at 5000 rpm for 10 min (Eppendorf 5415R). Subsequently, a pellet containing the bacteria was resuspended in a sample volume of PBS that was 10× the volume of the pellet. The resuspended cells were sonicated 5 times for 30 seconds using a Branson Sonic Power Company B12 Sonifier at maximal setting. Between the sonication steps, samples were cooled at 0° C. After the sonication treatment, intact bacterial cells were removed by centrifugation at 1000 g for 5 minutes at 4° C. (Eppendorf 5415R). The resulting R. pickettii extract was dissolved in Freund's incomplete adjuvant (for subcutaneous/intraperitoneal use) or 3% NaHCO₃ buffer (for oral use).

### Reversibility Experiments Using Antibody Treatment and Vaccination

Starting at the age of 10 weeks either anti-Ralstonia antibody (rabbit polyclonal antibody commercially produced by Thermo Fisher) was administrated either subcutaneously or intraperitoneally (10 μg/kg) once a week for 6 weeks, followed by daily Ralstonia pickettii gavage at week 12. In addition, another group of mice was treated at age of 10-11 weeks with Ralstonia vaccination comprising sonicated R. pickettii extract at 200 μg 10⁹ CFU intraperitoneally once weekly in incomplete Freund's adjuvant or 200 μg 10⁹ CFU R. pickettii extract in 300 μl 3% sodium hydrogen carbonate buffer was administered intragastrically at week 10 and 11 of age followed by daily Ralstonia pickettii gavage at week 12 (23). At week 17 oral glucose tolerance tests were repeated and animals were sacrificed. All experiments were approved by the Institutional Animal Care and Use Committee.

### Results

### Isolation of Bacterial DNA in Human Visceral Adipose Tissue

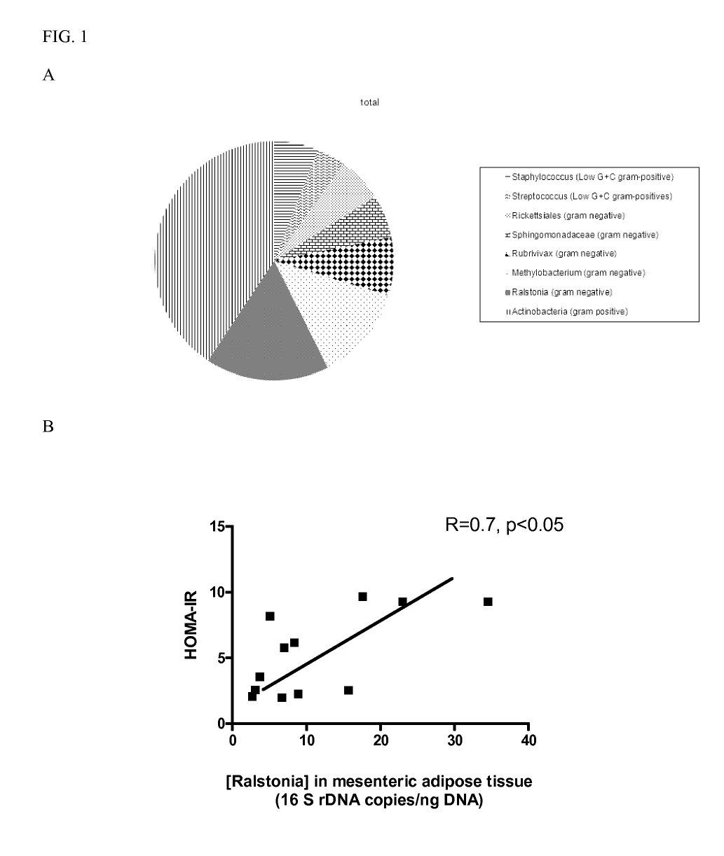

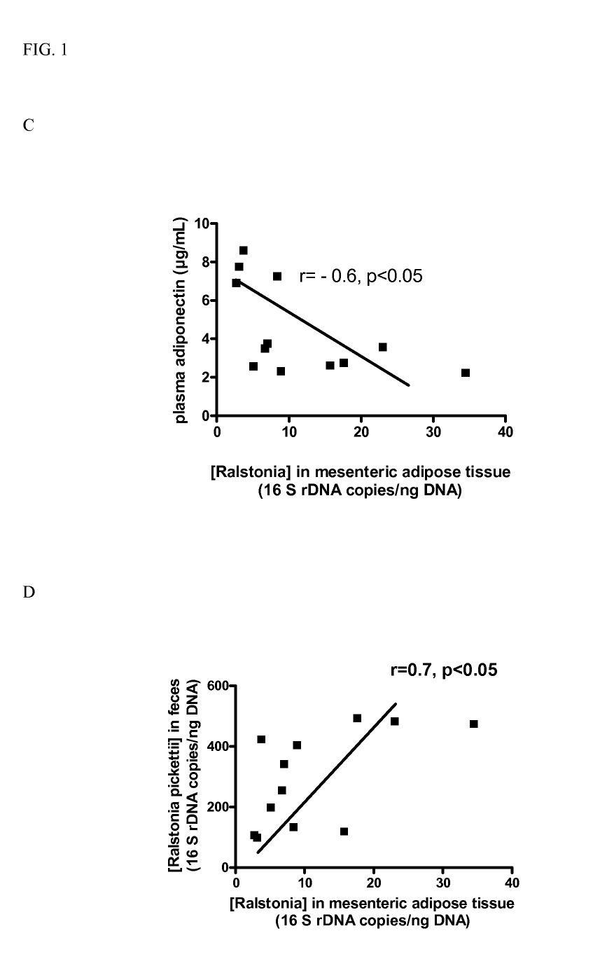

cDNA from visceral (mesenteric), omental and subcutaneous adipose tissue specimen (acquired under sterile conditions) was isolated. Bacterial DNA was only detected in visceral (mesenteric), and omental specimens using DGGE. Upon cloning and subsequent Sanger sequencing we identified 7 related bacteria of which the Gram-negative Ralstonia species was found to be the ubiquitous bacterial species in visceral-mesenteric adipose tissue (FIG. 1).

In addition to 16S DNA belong to the Gram-positive Actinobacteria, also two other Gram-positive bacterial genera, Staphylococcus and Streptococcus, were found, whereas 16S DNA of 6 Gram-negative bacterial taxa were detected (including the genera Ralstonia, Methylobacterium, Rubrivivax as well as Sphingomonadaceae, Rickettsiales and Actinobacteria) with Ralstonia being the most abundant Gram-negative. Ralstonia is a flagella-bearing Gram-negative facultative (an)aerobic rod (belonging to the Proteobacteria phylum), known to reside in the human intestine as a pathogen (22). We thus performed faecal qPCR for the 4 known Ralstonia strains associated with human disease (R. insidiosa, R. eutropha, R. pickettii and R. mannitolilytica) (22). Interestingly, we detected only signals of R pickettii and R. mannitolilytica in the faeces of these 12 subjects. Only the abundance of Ralstonia spp. in mesenteric adipose tissue correlated strongly with HOMA-IR (r=0.7, p<0.05) and showed an inverse relation (r=−0.6, p<0.05) with plasma adiponectin levels, an important marker of adipose tissue inflammation (FIGS. 1B and 1C). A significant correlation between faecal R. pickettii concentration and visceral adipose tissue Ralstonia content was found (r=0.7, P<0.05; FIG. 1D) underscoring that Ralstonia pickettii is the most abundant intestinal Ralstonia species to be present in mesenteric visceral adipose tissue.

Association Between Faecal Ralstonia pickettii and Development of Insulin Resistance

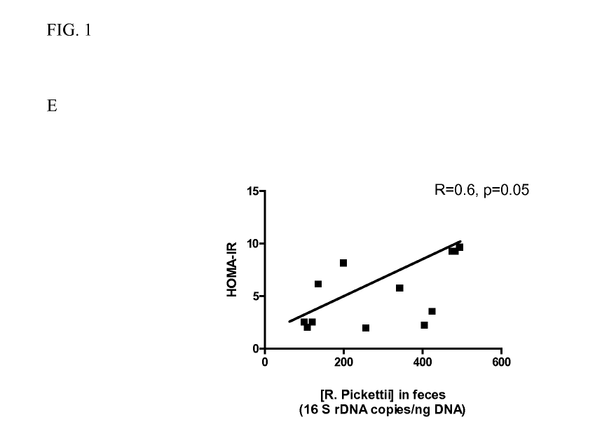

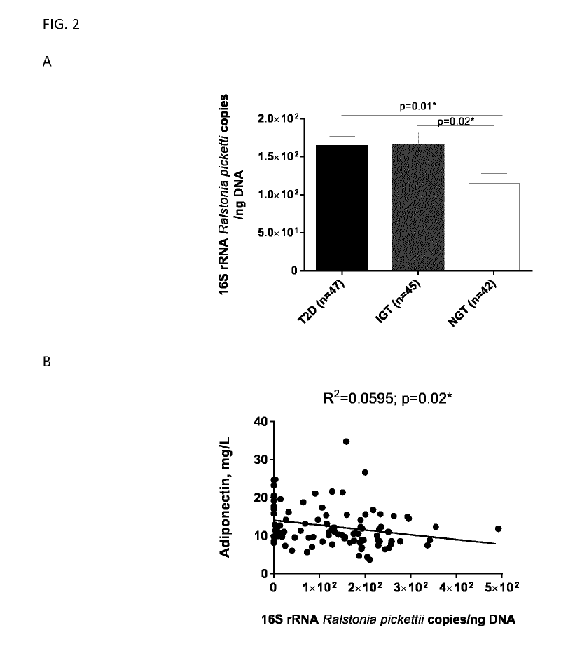

To study the association of faecal Ralstonia pickettii with development of insulin resistance and DM2, we used the previously described Swedish DIWA cohort to study the correlation between Ralstonia species and development of insulin resistance in a cohort of 134 obese postmenopausal Swedish females (8). We found that only Ralstonia pickettii could be detected in faeces; levels of Ralstonia pickettii were significantly increased in obese subjects that developed impaired fasting glucose (impaired glucose tolerance (IGT); n=45) and overt DM2 (type 2 diabetes (T2D); n=47) compared to controls (normal glucose tolerance (NGT); n=42) (see FIG. 2A). Moreover, we found that in the subjects with ranging levels of insulin resistance, plasma adiponectin was also inversely related to faecal Ralstonia pickettii levels (see FIG. 2B).

Causal Role of Ralstonia pickettii in Insulin Resistance

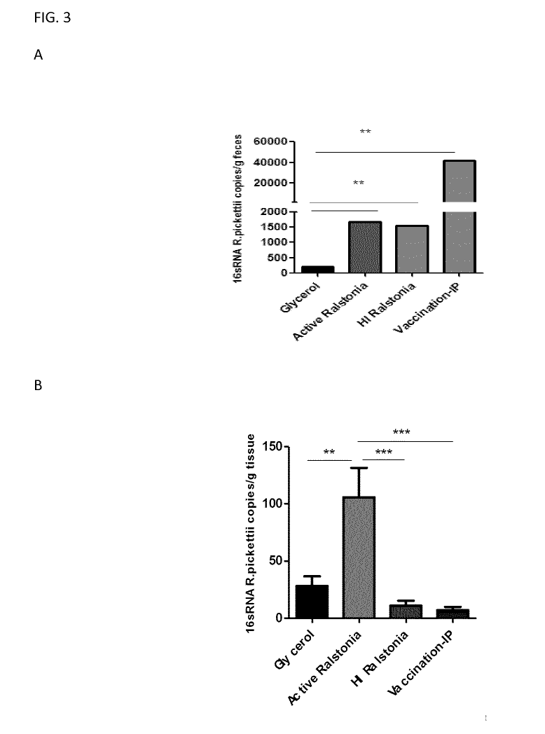

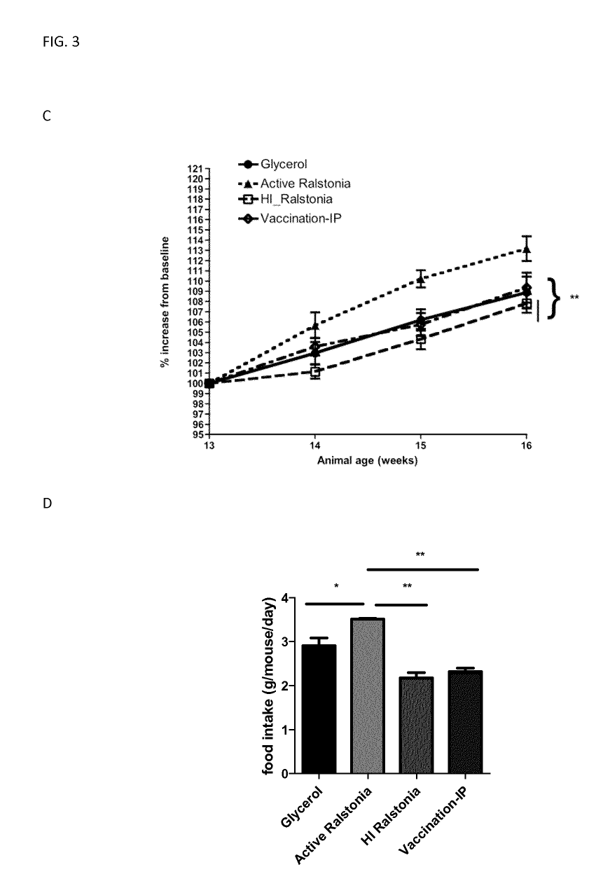

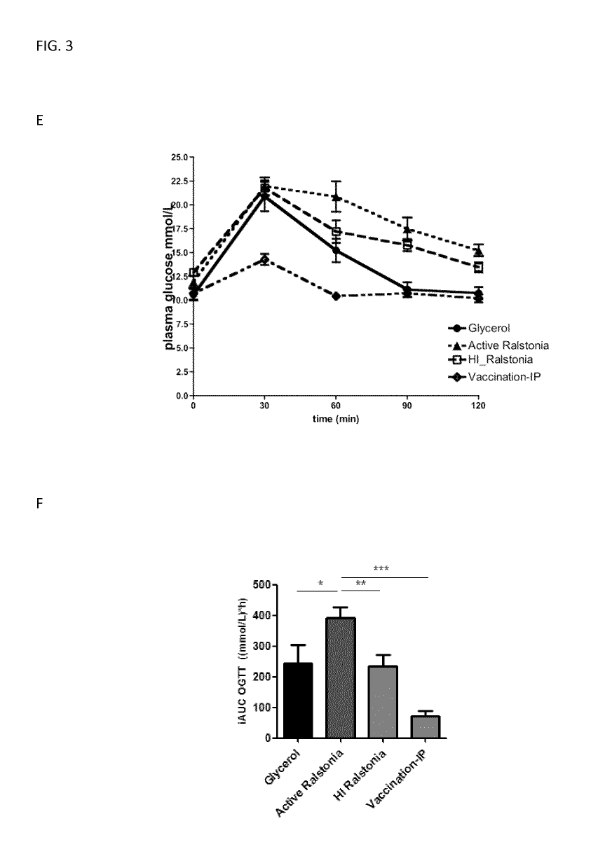

To examine potential causality in the development of obesity and insulin resistance and to study the effect of pre-vaccination against R. pickettii, we performed daily gavage of DIO mice with R. pickettii for 4 weeks. Daily oral gavage significantly increased fecal and mesenteric adipose tissue R. pickettii DNA levels compared to glycerol and heat-inactivated control (see FIGS. 3A and B). Moreover, pre-vaccination resulted in a significant increase in total bacterial load and intestinal R. pickettii upon daily inoculation, but no increase in mesenteric adipose tissue R. pickettii suggesting reduced bacterial translocation. Body weight and daily food intake were significantly increased in R. pickettii treated DIO mice, whereas pre-vaccination completely abrogated these metabolic derangements (FIGS. 3C and D). Alive R. pickettii significantly impaired glucose tolerance in DIO mice compared to DIO mice treated with heat-inactivated R. pickettii or glycerol. Similarly, pre-vaccination with R. pickettii extract completely abrogated R. pickettii-induced glucose intolerance (FIGS. 3E and F). Finally, as a marker of innate immune system activation, we investigated the effect of R. pickettii administration on gene expression levels of Toll-like receptors (Tlr) in mesenteric adipose tissue. TLRs are pattern recognition receptors (PPRs) that interact with bacterial cell wall components and can subsequently induce an inflammatory response. Tlr5 expression was significantly upregulated in mesenteric adipose tissue of mice treated with alive R. pickettii compared to heat-inactivated R. pickettii or glycerol-treated controls. In addition, R. pickettii pre-vaccination resulted in a decrease of Tlr4 and Tlr5 expression in mesenteric visceral adipose tissue.

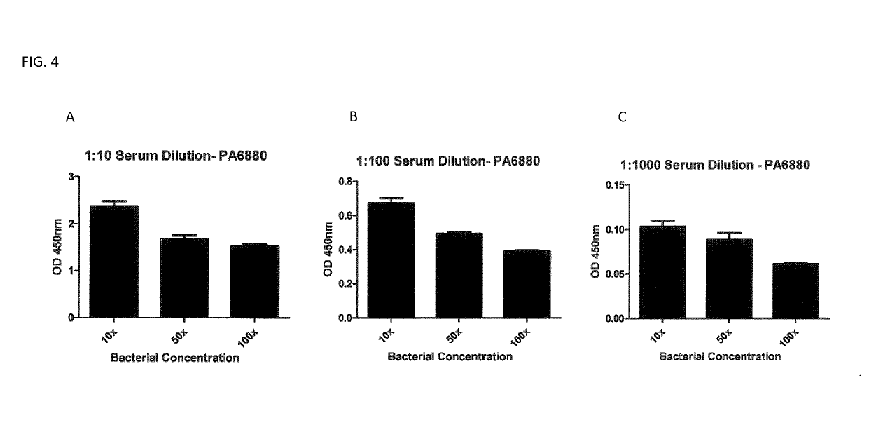

Finally, to study reversibility, we performed passive (Ralstonia antibody IP) boosting of the innate immune system and investigated effects on insulin resistance in DIO mice. Immunoboosting against intestinal Ralstonia pickettii significantly affected level of insulin resistance and subsequent visceral-mesenteric adipose tissue inflammation in DIO mice (FIG. 4).

In conclusion, the translational study described above reveals the identification of the Gram-negative Ralstonia species as most prevalent in human mesenteric visceral adipose tissue. Moreover, increased Ralstonia bacterial load in faeces of obese subjects predicted development of insulin resistance in a well phenotyped cohort of obese subjects. Finally, Ralstonia infection induced insulin resistance, whereas treatment with both polyclonal antibodies and vaccination against Ralstonia reversed this phenotype.

Effect of R. pickettii on Liver Genes Involved in NAFLD/NASH

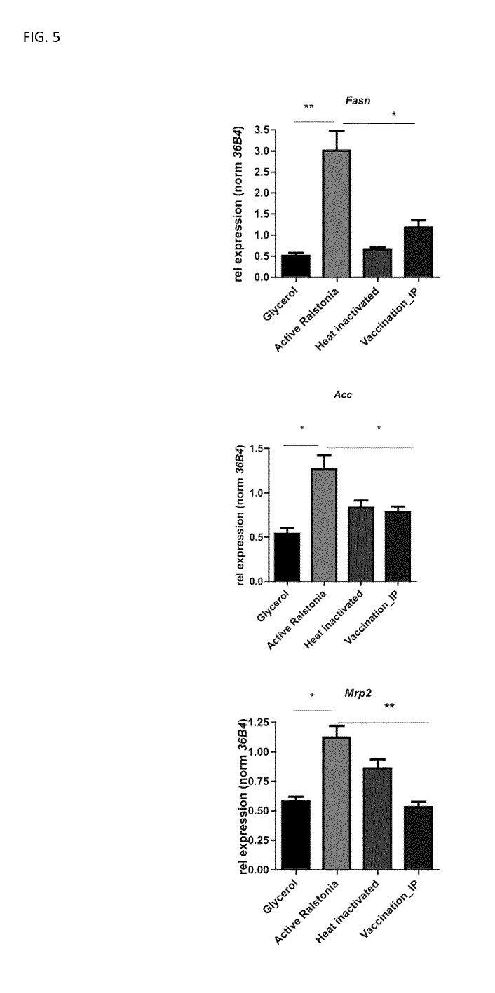

We subsequently studied whether liver genes involved in NAFLD/NASH development were altered upon Ralstonia pickettii vaccination. Thus, we analyzed expression of genes involved in liver fat metabolism including Acetyl CoA (ACC), Fatty acid synthase (FASN, a gene involved in synthesis of long-chain saturated fatty acids from acetyl-CoA, see Figarola JL PLoS One. 2013; 8(12):e83801) and MRP2 (a gene involved in development of NAFLD/NASH, see Sookoian S Nutr Biochem. 2009 October; 20(10):765-70). All three genes are key regulators of hepatic triglyceride (fat) storage and associated with development of NAFLD/NASH. It was found that 4 weeks of daily active Ralstonia pickettii gavage treatment significantly increased expression of Acetyl CoA (ACC), Fatty acid synthase (FASN) and ATP Binding Cassette Protein C2 (MRP2) in liver tissue of DIO mice. Conversely, DIO mice that were pretreated with Ralstonia pickettii intraperitoneal vaccination also normalized the expression of these genes as seen in the glycerol or heat inactivated control daily gavage treatment group (see FIG. 5).

It can be concluded that R. pickettii vaccination has beneficial effects on glucose metabolism, visceral adipose tissue inflammation and NAFLD/NASH in DIO mice.

### REFERENCES

1. Cecchini M, Sassi F, Lauer J A, Lee Y Y, Guajardo-Barron V, Chisholm D (2010) Tackling of unhealthy diets, physical inactivity, and obesity: health effects and cost-effectiveness. Lancet 376: 1775-1784
2. Kramer C K, Zinman B, Retnakaran R. Are metabolically healthy overweight and obesity benign conditions?: A systematic review and meta-analysis. Ann Intern Med. 2013; 159(11):758-69.
3. Fagerberg, B., Kellis, D., Bergstrom, G. & Behre, C. J. Adiponectin in relation to insulin sensitivity and insulin secretion in the development of type 2 diabetes: a prospective study in 64-year-old women. J Intern Med 2011 269, 636-643
4. Apovian C M, Bigornia S, Mott M, et al (2008) Adipose macrophage infiltration is associated with insulin resistance and vascular endothelial dysfunction in obese subjects. Arterioscler. Thromb. Vasc. Biol. 28: 1654-1659
5. Wentworth J M, Naselli G, Brown W A, et al (2010) Pro-inflammatory CD11c+CD206+ adipose tissue macrophages are associated with insulin resistance in human obesity. Diabetes 59: 1648-1656
6. Furet JP1, Kong L C, Tap J, Poitou C, Basdevant A, Bouillot J L, Mariat D, Corthier G, Doré J, Henegar C, Rizkalla S, Clément K. Differential adaptation of human gut microbiota to bariatric surgery-induced weight loss: links with metabolic and low-grade inflammation markers. Diabetes. 2010 December; 59(12):3049-57.
7. Le Chatelier et al. Richness of human gut microbiome correlates with metabolic markers. Nature. 2013 Aug. 29; 500(7464):541-6.
8. Karlsson F H, Tremaroli V, Nookaew I, Bergstrom G, Behre C J, Fagerberg B, Nielsen J, Backhed F. Gut metagenome in European women with normal, impaired and diabetic glucose control. Nature. 2013 Jun. 6; 498(7452):99-103.
9. Cani P D, Bibiloni R, Knauf C, et al (2008) Changes in gut microbiota control metabolic endotoxemia-induced inflammation in high-fat diet-induced obesity and diabetes in mice. Diabetes 57: 1470-1481
10. Clemente-Postigo M1, Queipo-Ortuño M I, Murri M, Boto-Ordoñez M, Perez-Martinez P, Andres-Lacueva C, Cardona F, Tinahones F J. Endotoxin increase after fat overload is related to postprandial hypertriglyceridemia in morbidly obese patients. J Lipid Res. 2012 May; 53(5):973-8.
11. Harte A L, Varma M C, Tripathi G, et al (2012) High fat intake leads to acute postprandial exposure to circulating endotoxin in type 2 diabetic subjects. Diabetes Care 35: 375-382
12. Sun L, Yu Z, Ye X, et al (2010) A marker of endotoxemia is associated with obesity and related metabolic disorders in apparently healthy Chinese. Diabetes Care 33: 1925-1932
13. Gummesson A1, Carlsson L M, Storlien L H, Backhed F, Lundin P, Löfgren L, Stenlöf K, Lam Y Y, Fagerberg B, Carlsson B. Intestinal permeability is associated with visceral adiposity in healthy women. Obesity (Silver Spring). 2011 November; 19(11):2280-2.
14. Amar J, Chabo C, Waget A, et al (2011) Intestinal mucosal adherence and translocation of commensal bacteria at the early onset of type 2 diabetes: molecular mechanisms and probiotic treatment. EMBO Mol. Med. 3: 559-572
15. Falkow S. Molecular Koch's postulates applied to bacterial pathogenicity—a personal recollection 15 years later. Nat Rev Microbiol. 2004 January; 2(l):67-72.
16. Zoetendal E G, von W A, Vilpponen-Salmela T, Ben-Amor K, Akkermans A D, de Vos W M (2002) Mucosa-associated bacteria in the human gastrointestinal tract are uniformly distributed along the colon and differ from the community recovered from faeces. Appl. Environ. Microbiol. 68: 3401-3407
17. Rajilic-Stojanovic M, Heilig H G, Molenaar D, et al (2009) Development and application of the human intestinal tract chip, a phylogenetic microarray: analysis of universally conserved phylotypes in the abundant microbiota of young and elderly adults. and differ from the community recovered from faeces. Appl. Environ. Microbiol. 68: 3401-3407
18. Zoetendal E G, Akkermans A D, de Vos W M (1998) Temperature gradient gel electrophoresis analysis of 16S rRNA from human faecal samples reveals stable and host specific communities of active bacteria. Appl. Environ. Microbiol. 64: 3854-3859
19. Heilig H G, Zoetendal E G, Vaughan E E, Marteau P, Akkermans A D, de Vos W M (2002) Molecular diversity of Lactobacillus spp. and other lactic acid bacteria in the human intestine as determined by specific amplification of 16S ribosomal DNA. Appl. Environ. Microbiol. 68: 114-123
20. Bergmark L, Poulsen P H, Al-Soud W A, Norman A, Hansen L H, Sorensen S J (2012) Assessment of the specificity of Burkholderia and Pseudomonas qPCR assays for detection of these genera in soil using 454 pyrosequencing. FEMS Microbiol. Lett. 333: 77-84
21. Zwielehner J, Lassl C, Hippe B, et al (2011) Changes in human faecal microbiota due to chemotherapy analyzed by TaqMan-PCR, 454 sequencing and PCR-DGGE fingerprinting. PLoS. One. 6: e28654
22. Ryan M P, Adley C C. Ralstonia spp.: emerging global opportunistic pathogens. Eur J Clin Microbiol Infect Dis. 2014; 33(3):291-304.
23. Guo L, Liu K, Zhao W, Li X, Li T, Tang F, Zhang R, Wu W, Xi T. Immunological features and efficacy of the reconstructed epitope vaccine CtUBE against Helicobacter pylori infection in BALB/c mice model. Appl Microbiol Biotechnol. 2013 March; 97(6):2367-78.
24. Caricilli A M, Picardi P K, de Abreu L L, Ueno M, Prada P O, Ropelle E R, Hirabara S M, Castoldi Â, Vieira P, Camara N O, Curi R, Carvalheira J B, Saad M J (2011). Gut microbiota is a key modulator of insulin resistance in TLR 2 knockout mice. PLoS Biol. 9(12):e1001212.
25. Vijay-Kumar M, Aitken J D, Carvalho F A, Cullender T C, Mwangi S, Srinivasan S, Sitaraman S V, Knight R, Ley R E, Gewirtz A T (2010). Metabolic syndrome and altered gut microbiota in mice lacking Toll-like receptor 5. Science. 328(5975):228-31
26. Shi H, Kokoeva M V, Inouye K, Tzameli I, Yin H, Flier JS.TLR4 links innate immunity and fatty acid-induced insulin resistance. J Clin Invest. 2006 November; 116(11):3015-25
27. Shulzhenko N, Morgun A, Hsiao W, et al (2011) Crosstalk between B lymphocytes, microbiota and the intestinal epithelium governs immunity versus metabolism in the gut. Nat. Med. 17: 1585-1593.
28. Vrieze A, van Nood E, Holleman F, et al (2012) Transfer of Intestinal Microbiota from Lean Donors Increases Insulin Sensitivity in Subjects with Metabolic Syndrome. Gastroenterology 143: 913-916.
29. Cullender TC1, Chassaing B, Janzon A, Kumar K, Muller C E, Werner J J, Angenent L T, Bell M E, Hay A G, Peterson D A, Walter J, Vijay-Kumar M, Gewirtz A T, Ley R E. Innate and adaptive immunity interact to quench microbiome flagellar motility in the gut. Cell Host Microbe. 2013 Nov. 13; 14(5):571-81
30. Vaneechoutte M, Kampfer P, De B T, Falsen E, Verschraegen G (2004) Wautersia gen. nov., a novel genus accommodating the phylogenetic lineage including Ralstonia eutropha and related species, and proposal of Ralstonia [Pseudomonas] syzygii comb. nov. Int. J.Syst. Evol. Microbiol. 54: 317-327
31. Moissenet D, Vu-Thien H, Benzerara Y, Arlet G (2003) DNA fingerprinting of Ralstonia paucula by infrequent-restriction-site PCR and randomly amplified polymorphic DNA analysis. J. Clin. Microbiol. 41: 5747-5749
32. Audfray A, Claudinon J, Abounit S, et al (2012) Fucose-binding lectin from opportunistic pathogen Burkholderia ambifaria binds to both plant and human oligosaccharidic epitopes. J. Biol. Chem. 287: 4335-4347
33. Shaw R A, Kryston L J, Mills L C (1968) Plasma fucose and fucose tolerance in diabetes mellitus. Ann. N.Y.Acad.Sci. 148: 787-795
34. Pacheco A R, Curtis M M, Ritchie J M, et al (2012) Fucose sensing regulates bacterial intestinal colonization. Nature 492: 113-117
35. Amar J, Serino M, Lange C, et al (2011) Involvement of tissue bacteria in the onset of diabetes in humans: evidence for a concept. Diabetologia 54: 3055-3061

1. A compound effective against Ralstonia pickettii for use in the treatment or prevention of insulin resistance, obesity, type II diabetes, metabolic syndrome, non-alcoholic fatty liver disease (NAFLD) or Non-alcoholic Steatosis Hepatitis (NASH) of a subject.

2. The compound for use according to claim 1, which is selected from the group consisting of:
a) an antigen derived from Ralstonia pickettii which is capable of inducing an immune response to a Ralstonia pickettii infection in a subject,
b) an antibody which is capable of neutralizing Ralstonia pickettii in an animal
c) an antibiotic effective against Ralstonia pickettii, and
d) a bacteriophage specific for Ralstonia pickettii.

3. A composition comprising a compound effective against Ralstonia pickettii as defined in claim 1 and a pharmaceutically-acceptable excipient, carrier, or diluent for use in the treatment or prevention of insulin resistance, obesity, type II diabetes, metabolic syndrome, non-alcoholic fatty liver disease (NAFLD) or Non-Alcoholic Steatosis Hepatitis (NASH) of a subject.

4. A vaccine composition suitable for treatment of an animal comprising a Ralstonia pickettii bacterium which is live, inactivated, or attenuated, or a fragment thereof capable of producing an immune response in a subject, and a pharmaceutically-acceptable excipient, carrier, or diluent.

5. The vaccine composition according to claim 4, wherein the fragment is selected from the group consisting of outer membrane fragments, outer membrane vesicles, outer membrane proteins or soluble variants thereof, or Ralstonia pickettii polysaccharides such as capsular polysaccharides and lipopolysaccharides.

6. The vaccine composition according to claim 4 for use as a medicament.

7. The vaccine composition according to claim 4 for use in the treatment or prevention of insulin resistance, obesity, type II diabetes, metabolic syndrome, non-alcoholic fatty liver disease (NAFLD) or Non-alcoholic Steatosis Hepatitis (NASH) of a subject.

8. An in vitro method of diagnosis or prediction of a disease or condition selected from insulin resistance, obesity, type II diabetes, metabolic syndrome, non-alcoholic fatty liver disease (NAFLD) or Non-alcoholic Steatosis Hepatitis (NASH), in a subject, comprising steps of:
a) determining the level of Ralstonia pickettii in a test sample derived from said subject,
b) comparing the level of Ralstonia pickettii in said test sample with a reference value or with the level of Ralstonia pickettii in a control sample, and
c) diagnosing or predicting said disease based on said comparison.

9. An in vitro method of diagnosis or prediction of a disease or condition selected from insulin resistance, obesity, type II diabetes, metabolic syndrome, non-alcoholic fatty liver disease (NAFLD) or Non-alcoholic Steatosis Hepatitis (NASH), in a subject, comprising steps of:
a) determining the level of an antibody capable of neutralizing Ralstonia pickettii in a test sample derived from said subject,
b) comparing the level of said antibody capable of neutralizing Ralstonia pickettii in said test sample with a reference value or with the level of said antibody capable of neutralizing Ralstonia pickettii in a control sample, and
c) diagnosing or predicting said disease based on said comparison.

10. The method according to claim 8, wherein said subject suffers from visceral fat inflammation.

11. The method according to claim 8, wherein said test sample is selected from the group consisting of a faeces sample or a sample comprising adipose cells, preferably from visceral adipose fat.

12. The method according to claim 9, wherein said test sample is a blood sample.

13. The method according to claim 8, which comprises determining the level of a nucleic acid of Ralstonia pickettii in said test sample, preferably wherein said nucleic acid is 16S rRNA.

14. The method according to claim 8, wherein an increased level of R. pickettii in said test sample compared to said control sample is indicative of a diagnosis or prediction of a disease or condition selected from insulin resistance, obesity, type II diabetes, metabolic syndrome, non-alcoholic fatty liver disease (NAFLD) or Non-alcoholic Steatosis Hepatitis (NASH), in said subject.

15. The method according to claim 9, wherein an increased level of said antibody capable of neutralizing Ralstonia pickettii in said test sample compared to said control sample is indicative of a diagnosis or prediction of a disease or condition selected from insulin resistance, obesity, type II diabetes, metabolic syndrome, non-alcoholic fatty liver disease (NAFLD) or Non-alcoholic Steatosis Hepatitis (NASH), in said subject.

16. Use of a compound selected from the group consisting of an antibody capable of neutralizing Ralstonia pickettii in an animal, a Ralstonia pickettii cell or a part thereof, and/or a nucleic acid hybridizing under stringent conditions to a nucleic acid from Ralstonia pickettii, in a method according to claim 8.

17. Use of a kit comprising at least one compound as defined in claim 16, and optionally comprising a further reagent or a conventional kit component in a method according claim 8.

18. A composition comprising a compound effective against Ralstonia pickettii as defined in claim 2 and a pharmaceutically-acceptable excipient, carrier, or diluent for use in the treatment or prevention of insulin resistance, obesity, type II diabetes, metabolic syndrome, non-alcoholic fatty liver disease (NAFLD) or Non-Alcoholic Steatosis Hepatitis (NASH) of a subject.

19. The vaccine composition according to claim 5 for use as a medicament.

20. The vaccine composition according to claim 5 for use in the treatment or prevention of insulin resistance, obesity, type II diabetes, metabolic syndrome, non-alcoholic fatty liver disease (NAFLD) or Non-alcoholic Steatosis Hepatitis (NASH) of a subject.

21. The method according to claim 9, wherein said subject suffers from visceral fat inflammation.

22. The method according to claim 9, which comprises determining the level of a nucleic acid of Ralstonia pickettii in said test sample, preferably wherein said nucleic acid is 16S rRNA.

23. The method according to claim 10, which comprises determining the level of a nucleic acid of Ralstonia pickettii in said test sample, preferably wherein said nucleic acid is 16S rRNA.

24. The method according to claim 11, which comprises determining the level of a nucleic acid of Ralstonia pickettii in said test sample, preferably wherein said nucleic acid is 16S rRNA.
